(19) United States
(12) Patent Application Publication
(10) Pub. No.: US 2026/0240961 A1
(43) Pub. Date: Aug. 20, 2026

(54) NUTRITIONAL COMPOSITIONS COMPRISING A RECOMBINANT POLYPEPTIDE WHICH HAS A NUTRITIONALLY COMPLETE AMINO ACID PROFILE

(71) Applicant: NOVOZYMES A/S, Bagsvaerd (DK)

(72) Inventors: Jesper VIND, Vaerloese (DK); Morten GJERMANSEN, Greve (DK); Birgitte ANDERSEN, Baagsvaerd (DK)

(73) Assignee: NOVOZYMES A/S, Bagsvaerd (DK)

(21) Appl. No.: 19/159,316

(22) PCT Filed: Feb. 29, 2024
(86) PCT No.: PCT/EP2024/055169

§ 371 Date: Aug. 24, 2025

(30) Foreign Application Priority Data
Mar. 2, 2023 (EP) 23159538.0

(51) Int. Cl. A61K 38/47; C12N 9/24

(52) U.S. Cl. CPC A61K 38/47; C12N 9/2402; C12Y 302/01025

(57) ABSTRACT

The invention provides a nutritional composition comprising a recombinant polypeptide that has a nutritionally complete amino acid profile suitable for daily human consumption for general nutrition and specialized nutrition. The invention further provides a recombinant polypeptide and variants and truncates thereof which are suitable to provide a nutritionally complete amino acid profile when provided in a nutritional composition. The invention also provides polynucleo-tides encoding the recombinant polypeptides, nucleic acid constructs, vectors, and host cells comprising the polynucleotides, and methods of producing said recombinant polypeptides.

### REFERENCE TO A SEQUENCE LISTING

This application contains a Sequence Listing in computer readable form. The computer readable form is incorporated herein by reference.

### FIELD OF THE INVENTION

The present invention relates to nutritional compositions comprising a recombinant polypeptide of the invention, the recombinant polypeptides of the invention, polynucleotides encoding the recombinant polypeptides, nucleic acid constructs, vectors, and host cells comprising the polynucleotides, and methods of producing said recombinant polypeptides.

### BACKGROUND OF THE INVENTION

There is a general need for all human diets to comprise a dietary protein source which is of high quality. The body cannot synthesize certain amino acids that are necessary for health and growth. These “essential” amino acids are histidine, isoleucine, leucine, lysine, methionine, phenylalanine, threonine, tryptophan, and valine. Dietary proteins that provide all essential amino acids are referred to as high quality proteins, or complete proteins. Foods that have higher protein quality are considered more beneficial in a mammalian diet compared to proteins that do not. Complete proteins promote maintenance of muscle mass, a healthy body mass index, and glycemic balance. Additionally, by increasing the amount of complete protein in the diet, the total amount of protein consumption may be decreased compared to a diet in which low quality proteins are primarily consumed.

There is a general need for foods which are high in protein and have amino acid compositions that meet daily requirements for essential amino acids, for general nutrition. Additionally, there is a need for food which satisfy the protein needs for those requiring specialized nutrition. Specialized nutrition includes the nutritional needs of ill or elderly patients or patients with certain conditions such as pregnancy, short-term medical needs such as hospitalization, or long-term medical needs such as chronic conditions like diabetes or metabolic disorders.

Dietary products that are enriched with protein, either for general nutrition or specialized nutrition, are typically enriched with proteins that are relatively easy to access and low-cost to purify. Such proteins include whey proteins, casein, and albumin. Although these proteins are a good source of many essential amino acids, they cannot be tailored to provide desirable amounts of particular amino acids. Additionally, patients with metabolic disorders frequently require the exclusion of at least one essential amino acid from their diets and high enrichment of other amino acids. To address at least part of this problem, dietary products frequently comprise free amino acids which are added at amounts to provide a complete nutritional amino acid profile. Unfortunately, free amino acids have an extremely bitter taste which is not completely masked by the addition of other ingredients, such as sugars and/or flavorants. Therefore, these dietary products are not desirable to those who must consume them.

There is a need for a protein source which can be tailored to provide a desired nutritional profile and which can also be provided in a cost-effective manner.

### SUMMARY OF THE INVENTION

The invention provides a recombinant polypeptide which can be modified to be suitable for nutritional compositions as a complete protein source for general or specialized nutrition. For general nutritional compositions, the recombinant polypeptide may be modified to provide a variant(s) with a nutritionally complete amino acid profile, as determined by an Amino Acid Score of greater than or equal to 0.94. The invention also provides polypeptide variants that can be tailored for specialized nutrition by enriching and/or depleting certain amino acids, depending on the amino acid needs of the group for which the nutritional composition is targeted. Because the recombinant polypeptides of the invention comprise a complete nutritional profile, free amino acids do not need to be added to the composition. Therefore, the composition does not have the bitter taste associated with many dietary products available today. The recombinant polypeptides of the invention are highly expressed and relatively easy to purify, so that they are extremely cost-efficient to manufacture. Additionally, the recombinant polypeptides of the invention can tolerant broad pH ranges and high temperature treatments, so that they are compatible in a variety of foods and also in processes of food manufacturing.

### Definitions

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

This disclosure makes reference to amino acids. The full name of the amino acids is used interchangeably with the standard three letter and one letter abbreviations for each. For the avoidance of doubt, those are Alanine (Ala, A), Arginine (Arg, R), Asparagine (Asn, N), Aspartic acid (Asp, D), Cysteine (Cys, C), Glutamic Acid (Glu, E), Glutamine (Gln, Q), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), Valine (Val, V).

Amino acid composition: The amino acid composition of a polypeptide is calculated as the milligram amino acid per gram polypeptide. Amino acid composition may be calculated for a particular amino acid of interest, such as for example the amount of leucine in a given polypeptide, or for a group of amino acids, such as the amount of branched chain amino acids in a given polypeptide. Calculation may be based on the known sequence of the polypeptide, for example by the total molecular weight of the amino acid residues of interest divided by the total molecular weight of the polypeptide. The total molecular weight of the amino acid residues of interest is determined by the number of said residues in the polypeptide multiplied by the molecular weight of the residue in the context of the polypeptide (as opposed to the molecular weight of the free amino acid).

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the variant, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: An “expression vector” refers to a linear or circular DNA construct comprising a DNA sequence encoding a variant, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a polypeptide.

Fragment: The term “fragment” means a variant having one or more amino acids absent from the amino and/or carboxyl terminus of the polypeptide.

Fusion polypeptide: The term “fusion polypeptide” is a polypeptide in which one polypeptide is fused at the N-terminus and/or the C-terminus of a variant of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a variant has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Improved property: The term “improved property” means a characteristic associated with a variant that is improved compared to the parent.

Introduced: The term “introduced” in the context of inserting a nucleic acid sequence into a cell, means “transfection”, “transformation” or “transduction,” as known in the art.

Isolated: The term “isolated” means a variant, nucleic acid, cell, or other specified material or component that is separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted variant expressed in a host cell.

Mature polypeptide: The term “mature polypeptide” means a polypeptide in its mature form following N-terminal processing and/or C-terminal processing (e.g., removal of signal peptide).

Mature polypeptide coding sequence: The term “mature polypeptide coding sequence” means a polynucleotide that encodes a mature polypeptide.

Mutant: The term “mutant” means a polynucleotide encoding a variant.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a variant. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Parent or parent polypeptide: The term “parent” or “parent polypeptide” means a polypeptide to which an alteration is made to produce the polypeptide variants of the present invention.

Purified: The term “purified” means a nucleic acid, variant or cell that is substantially free from other components as determined by analytical techniques well known in the art (e.g., a purified variant or nucleic acid may form a discrete band in an electrophoretic gel, chromatographic eluate, and/or a media subjected to density gradient centrifugation). A purified nucleic acid or variant is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, variant, cell, nucleic acid, amino acid, or other specified material or component that is present in a composition at a relative or absolute concentration that is higher than a starting composition.

In one aspect, the term “purified” as used herein refers to the variant or cell being essentially free from components (especially insoluble components) from the production organism. In other aspects, the term “purified” refers to the variant being essentially free of insoluble components (especially insoluble components) from the native organism from which it is obtained. In one aspect, the variant is separated from some of the soluble components of the organism and culture medium from which it is recovered. The variant may be purified (i.e., separated) by one or more of the unit operations filtration, precipitation, or chromatography.

Accordingly, the variant may be purified such that only minor amounts of other proteins, in particular, other polypeptides, are present. The term “purified” as used herein may refer to removal of other components, particularly other proteins and most particularly other enzymes present in the cell of origin of the polypeptide. The variant may be “substantially pure”, i.e., free from other components from the organism in which it is produced, e.g., a host organism for recombinantly produced variant. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure variant is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The variant of the present invention is preferably in a substantially pure form (i.e., the preparation is essentially free of other polypeptide material with which it is natively or recombinantly associated). This can be accomplished, for example by preparing the variant by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, variant, polypeptide, or vector that has been modified from its native state. Thus, for example, recombinant cells express genes that are not found within the native (non-recombinant) form of the cell, or express native genes at different levels or under different conditions than found in nature. A recombinant polypeptide is the product of these genes expressed in a recombinant cell. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g., depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hyrdo cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence.

Truncate: The term “truncate” means a polypeptide that is smaller than the full-length polypeptide. The truncate may be truncated at the C-terminal end, the N-terminal end, or both. The truncate may also comprise or in alternative comprise an internal truncation, where an internal portion(s) of the full-length sequence may be removed.

Variant: The term “variant” means a polypeptide comprising a substitution, an insertion (including extension), and/or a deletion (including truncation), at one or more positions compared to its parent. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

Sufficient amount: The term “sufficient amount” is an amount of a polypeptide or amino acid that is sufficient to cause the desired effect. A sufficient amount of a polypeptide or amino acid can be provided directly, i.e., by administering the polypeptide or amino acid to a subject, or it can be provided as part of a composition comprising the polypeptide or amino acid.

Amino Acid Score (AAS): The term “Amino Acid Score” (AAS) is based on essential amino acid requirements published by the Food and Agriculture Organization (FAO) for different age groups (“Dietary protein quality evaluation in human nutrition: Report of an FAO Expert Consultation”, FAO Food Nutr Paper, 92:1-66, 2013). In this report, scoring patterns for protein quality evaluation was determined by calculating the ratio of an essential amino acid to protein requirement, expressed as mg amino acid per g protein. Using the scoring pattern determined for the protein requirements for a child (6 months to 3 years; see Table 5 of the referenced FAO report), the AAS was defined. To calculate the AAS for a given polypeptide, the ratio of the amount of an essential amino acid present in the polypeptide compared to the amount of that essential amino acid recommended in the FAO report is determined, for each essential amino acid. The AAS of that polypeptide is the lowest ratio determined for any of the essential amino acids. A polypeptide with an AAS of greater than 0.94 satisfies the essential amino acid requirements for all essential amino acids and is generally considered a complete protein source.

Branched chain amino acid (BCAA): The term “branched chain amino acid” is an amino acid selected from leucine, isoleucine, and valine.

Essential amino acid: The term “essential amino acid” is an amino acid selected from histidine, isoleucine, leucine, lysine, methionine, phenylalanine, threonine, tryptophan, and valine.

Large neutral amino acid (LNAA): The term “large neutral amino acid” is an amino acid selected from phenylalanine, arginine, histidine, isoleucine, leucine, lysine, methionine, threonine, tryptophan, tyrosine and valine. These amino acids share the same transport system in the brain and intestinal mucosa.

Nutritional composition: The term “nutritional composition” is a composition comprising a recombinant polypeptide of the invention which contains a desirable amount of amino acids. The nutritional composition may also include any number of optional additional ingredients, including conventional food additives (synthetic or natural), for example one or more acidulants, additional thickeners, buffers or agents for pH adjustment, chelating agents, colorants, emulsifiers, excipient, flavorants, minerals, osmotic agents, acceptable carriers, preservatives, stabilizers, sugars, sweeteners, texturizers, minerals, and/or vitamins. The optional ingredients can be added in any suitable amount. The nutritional compositions may be a source of complete nutrition or may be a source of incomplete nutrition. The nutritional composition may provide daily dietary requirements for protein or for essential amino acids.

Complete nutrition: The term “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100 percent of their nutritional requirements from such complete nutritional compositions.

Effective amount: The term “effective amount” is an amount that prevents a deficiency, treats a disease or medical condition in an individual or, more generally, reduces symptoms, manages progression of a disease or provides a nutritional, physiological, or medical benefit to the individual. An effective amount may also be an amount that meets daily dietary requirements for protein or for essential amino acids for a generally healthy person based on published recommendations from the FAO (FAO Food Nutr Paper, 92:1-66, 2013).

Patient or individual: The term “patient” or “individual” are often used herein to refer to a human. However, in some embodiments, the terms “individual” and “patient” refer to any animal, mammal, or human having a medical condition that can benefit from a nutritional composition of the invention, or any animal, mammal or human that does not have a medical condition and can benefit from a nutritional composition of the invention for general health.

### Conventions for Designation of Variants

In describing the variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine(S) with phenylalanine (F), respectively.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or

“G195*+S411*”.

Insertions: For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations: Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations: Where different alterations can be introduced at a position, the different alterations are separated by a comma, e.g., “Arg170Tyr, Glu” represents a substitution of arginine at position 170 with tyrosine or glutamic acid. Thus, “Tyr167Gly, Ala+Arg170Gly, Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

A recombinant polypeptide was identified which was found to express at very high levels when expressed recombinantly in Aspergillus niger, Aspergillus oryzae, and Bacillus licheniformis, and also to purify to a high level relatively easily using methods well-known in the art. Surprisingly, this polypeptide is also suitable in a nutritional composition, as it possesses an amino acid composition which provides a value close to a desirable AAS and also does not contain either toxic domains or amino acid motifs known to play a role in protein allergens. Although the polypeptide possesses mannanase activity (described in WO2021/152123, incorporated by reference herein), an inactive variant can be produced by introducing a substitution at positions E426 and/or E334, using the amino acid sequence of SEQ ID NO: 49 for numbering. Examples of inactive variants are SEQ ID NO: 50 and 51. The inventors have found that an inactive variant still possesses high expression and ease of purification.

Additionally, variants of the mannanase polypeptide, which are polypeptides of the invention, tolerate heat treatment and a broad range of pH. This indicates they are suitable as components of nutritional compositions, which frequently have a neutral or acidic pH, and also are suitable to withstand production and processing of a nutritional composition, which may require heat during production and likely also require some form of heat treatment for sterilization.

The present invention further relates to a nutritional composition comprising variants of the mannanase polypeptide suitable as a complete or near complete protein source for daily human consumption. A nutritional composition of the invention may comprise variant(s) of the mannanase polypeptide suitable for individuals with increased medical needs and/or long term care, including the elderly, pregnant women, cancer patients, and individuals with long-term diseases such as diabetes. A nutritional composition of the invention may comprise variant(s) of the mannanase polypeptide suitable as a complete protein source for patients with a metabolic disorder.

The polypeptides of the present invention are recombinant and comprise SEQ ID NOs: 3 to 101 and any variant thereof. A variant has at least 60%, e.g., at least 65%, at least 70%, at least 75%, at least 80%, at least 85%, at least 90%, at least 91%, at least 93%, at least 94%, at least 95%, at least 96%, at least 97%, at least 98%, or at least 99% sequence identity, or at least 100% sequence identity, to the polypeptide of any one of SEQ ID NOs: 3 to 101.

A polypeptide of the invention may comprise a substitution, an insertion or a deletion at one or more positions of the polypeptide of any one of SEQ ID NOs: 3 to 101. A polypeptide of the invention may further comprise an extension of one or more amino acids at the N-terminal and/or C-terminal ends.

In some embodiments, a polypeptide of the invention may be a fusion polypeptide or a cleavable fusion polypeptide. A fusion polypeptide is produced by fusing a polynucleotide encoding another polypeptide to a polynucleotide encoding a polypeptide of the present invention. The fusion polypeptide may comprise a fragment of a polypeptide of the invention or a full-length polypeptide of the invention. In some embodiments, a the fusion polypeptide may comprise 490 to 450, 450 to 400, 400 to 350, 350 to 300, 300 to 250, 250 to 200, 200 to 150, 150 to 100, or 100 to 50 amino acids of any one of SEQ ID NOs: 3 to 101.

Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Alternatively, a polypeptide of the invention may be a truncated variant, comprising a truncation of one or more amino acids at the N-terminal and/or C-terminal ends, or an internal truncation where amino acids at the N-terminal or C-terminal are preserved and a number of amino acids internally are removed, relative to SEQ ID NO: 51. Polypeptides of the invention include a truncated variant of any one of SEQ ID NOs: 3 to 101. A polypeptide of the invention may be a truncate that comprises at least 100, at least 150, at least 200, at least 250, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470 or at least 480 consecutive amino acid residues of any one of SEQ ID NO: 3 to 101. In some embodiments, a polypeptide of the invention is a truncated variant comprising an amino acid sequence with at least 60%, at least 65%, at least 70%, at least 75%, at least 80%, at least 85%, at least 90%, at least 91%, at least 93%, at least 94%, at least 95%, at least 96%, at least 97%, at least 98%, or at least 99% sequence identity, or at least 100% sequence identity to a truncate of any one of SEQ ID NOs: 3 to 101.

In some embodiments, a polypeptide of the invention comprises an amino acid sequence with at least 60%, at least 65%, at least 70%, at least 75%, at least 80%, at least 85%, at least 90%, at least 95%, or 100% sequence identity to a truncate of amino acid sequence of SEQ ID NO: 3, wherein the truncate has an C-terminal truncation of 1-350 amino acids, 1-340 amino acids, 1-330 amino acids, 1-320 amino acids, 1-310 amino acids, 1-300 amino acids, 1-290 amino acids, 1-285 amino acids, 1-280 amino acids, 1-275 amino acids, 1-270 amino acids, 1-265 amino acids, 1-260 amino acids, 1-255 amino acids, 1-250 amino acids, 1-245 amino acids, 1-240 amino acids, 1-235 amino acids, 1-230 amino acids, 1-225 amino acids, 1-220 amino acids, 1-215 amino acids, 1-210 amino acids, 1-205 amino acids, 1-200 amino acids, 1-195 amino acids, 1-190 amino acids, 1-185 amino acids, 1-180 amino acids, 1-175 amino acids, 1-170 amino acids, 1-165 amino acids, 1-160 amino acids, 1-155 amino acids, 1-150 amino acids, 1-145 amino acids, 1-140 amino acids, 1-135 amino acids, 1-130 amino acids, 1-125 amino acids, 1-120 amino acids, 1-115 amino acids, 1-110 amino acids, 1-95 amino acids, 1-90 amino acids, 1-85 amino acids, 1-80 amino acids, 1-75 amino acids, 1-70 amino acids, 1-65 amino acids, 1-60 amino acids, 1-55 amino acids, 1-50 amino acids, 1-45 amino acids, 1-40 amino acids, 1-35 amino acids, 1-30 amino acids, 1-25 amino acids, 1-20 amino acids, 1-15 amino acids, 1-10 amino acids, or 1-5 amino acids relative to the amino acid sequence of SEQ ID NO: 51.

In some embodiments, a polypeptide of the invention comprises an amino acid sequence with at least 60%, at least 65%, at least 70%, at least 75%, at least 80%, at least 85%, at least 90%, at least 95%, or 100% sequence identity to a truncate of amino acid sequence of SEQ ID NO: 3, wherein the truncate has an N-terminal truncation of 1-350 amino acids, 1-340 amino acids, 1-330 amino acids, 1-320 amino acids, 1-310 amino acids, 1-300 amino acids, 1-290 amino acids, 1-285 amino acids, 1-280 amino acids, 1-275 amino acids, 1-270 amino acids, 1-265 amino acids, 1-260 amino acids, 1-255 amino acids, 1-250 amino acids, 1-245 amino acids, 1-240 amino acids, 1-235 amino acids, 1-230 amino acids, 1-225 amino acids, 1-220 amino acids, 1-215 amino acids, 1-210 amino acids, 1-205 amino acids, 1-200 amino acids, 1-195 amino acids, 1-190 amino acids, 1-185 amino acids, 1-180 amino acids, 1-175 amino acids, 1-170 amino acids, 1-165 amino acids, 1-160 amino acids, 1-155 amino acids, 1-150 amino acids, 1-145 amino acids, 1-140 amino acids, 1-135 amino acids, 1-130 amino acids, 1-125 amino acids, 1-120 amino acids, 1-115 amino acids, 1-110 amino acids, 1-95 amino acids, 1-90 amino acids, 1-85 amino acids, 1-80 amino acids, 1-75 amino acids, 1-70 amino acids, 1-65 amino acids, 1-60 amino acids, 1-55 amino acids, 1-50 amino acids, 1-45 amino acids, 1-40 amino acids, 1-35 amino acids, 1-30 amino acids, 1-25 amino acids, 1-20 amino acids, 1-15 amino acids, 1-10 amino acids, or 1-5 amino acids relative to the amino acid sequence of SEQ ID NO: 51.

In some embodiments, a polypeptide of the invention comprises an amino acid sequence with at least 60%, at least 65%, at least 70%, at least 75%, at least 80%, at least 85%, at least 90%, at least 95%, or 100% sequence identity to a truncate of amino acid sequence of SEQ ID NO: 3, wherein the truncate has an internal truncation of 1-350 amino acids, 1-340 amino acids, 1-330 amino acids, 1-320 amino acids, 1-310 amino acids, 1-300 amino acids, 1-290 amino acids, 1-285 amino acids, 1-280 amino acids, 1-275 amino acids, 1-270 amino acids, 1-265 amino acids, 1-260 amino acids, 1-255 amino acids, 1-250 amino acids, 1-245 amino acids, 1-240 amino acids, 1-235 amino acids, 1-230 amino acids, 1-225 amino acids, 1-220 amino acids, 1-215 amino acids, 1-210 amino acids, 1-205 amino acids, 1-200 amino acids, 1-195 amino acids, 1-190 amino acids, 1-185 amino acids, 1-180 amino acids, 1-175 amino acids, 1-170 amino acids, 1-165 amino acids, 1-160 amino acids, 1-155 amino acids, 1-150 amino acids, 1-145 amino acids, 1-140 amino acids, 1-135 amino acids, 1-130 amino acids, 1-125 amino acids, 1-120 amino acids, 1-115 amino acids, 1-110 amino acids, 1-95 amino acids, 1-90 amino acids, 1-85 amino acids, 1-80 amino acids, 1-75 amino acids, 1-70 amino acids, 1-65 amino acids, 1-60 amino acids, 1-55 amino acids, 1-50 amino acids, 1-45 amino acids, 1-40 amino acids, 1-35 amino acids, 1-30 amino acids, 1-25 amino acids, 1-20 amino acids, 1-15 amino acids, 1-10 amino acids, or 1-5 amino acids relative to the amino acid sequence of SEQ ID NO: 51.

SEQ ID NOs: 4-48, 52, 53, 58, 61, 62, 63, 66, 67, and 70-75 are truncated variants of SEQ ID NO: 3. In some embodiments, a truncate may be an N-terminal truncate where amino acids 1-265 are removed relative to SEQ ID NO: 3, such as for example SEQ ID NO: 10. In some embodiments, a truncate may be an N-terminal truncation where amino acids 1-158 are removed relative to SEQ ID NO: 3, such as for examples SEQ ID NOs: 4-8, 11-43, and 45-48. In some embodiments, a truncate may be an N-terminal truncation where amino acids 1-157 are removed relative to SEQ ID NO: 3, such as for examples SEQ ID NO: 9. In some embodiments, a truncate may possess the first 11 amino acids from the N-terminal and then an internal truncation from positions 12-160, with position numbering according to the amino acid sequence of SEQ ID NO: 3, such as for example internal truncate variants SEQ ID NO: 52 and 53.

Polypeptides of the invention are recombinant polypeptides. A polypeptide of the invention comprises an amino acid sequence with at least 60%, at least 65%, at least 70%, at least 75%, at least 80%, at least 85%, at least 90%, at least 95%, or 100% sequence identity to the amino acid sequence of SEQ ID NO: 3-101 or a truncate thereof, and further comprises a substitution at a position corresponding to position E334 and/or E426 of SEQ ID NO: 49. The substitution to position E334 and/or E426 may be an A, C, D, F, G, H, I, K, L, M, N, P, Q, R, S, T, V, W, or Y. In some embodiments, the substitution to position E334 and/or E426 may be an A, I, G, L, M, Q, V. In some embodiments, only position E334 has a substitution. In other embodiments, only position E426 has a substitution. In some embodiments, both position E334 and E426 are substituted.

### Amino Acid Composition

Polypeptides of the invention have an amino acid score (AAS) of greater than or equal to 0.94 (≥0.94). The AAS is based on essential amino acid requirements published by the Food and Agriculture Organization for different age groups (“Dietary protein quality evaluation in human nutrition: Report of an FAO Expert Consultation”, FAO Food Nutr Paper, 92:1-66, 2013), particularly on the scoring pattern for the protein requirements for a child (6 months to 3 years). To calculate the AAS for a given polypeptide, the ratio of the amount of an essential amino acid present in the polypeptide compared to the amount of that essential amino acid recommended in the FAO report is determined, for each essential amino acid. The AAS of that polypeptide is the lowest ratio determined for any of the essential amino acids. A polypeptide with an AAS of greater than or equal to 0.94 satisfies the essential amino acid requirements for all essential amino acids and is generally considered a complete protein source. In some embodiments, a polypeptide of the invention has an AAS greater than or equal to 0.94, 0.95, 0.96, 0.97, 0.98, 0.99, or 1.

A polypeptide suitable for a patient with a metabolic disorder is considered a complete protein source after the amino acid composition is adjusted for the needs of the patient. For example, Phenylketonuria (PKU) and Hyperphenylalaninemia (HPA) are inherited metabolic disorders in which phenylalanine (Phe) cannot be properly processed. Therefore, phenylalanine in the diet needs to be avoided as much as possible. A recombinant polypeptide of the invention suitable as a complete protein source for PKU or HPA patients has no phenylalanine residues, and other than phenylalanine has an AAS ≥0.94.

In addition to being deficient in certain amino acids, a recombinant polypeptide that is to serve as a major protein source for a patient with a metabolic disorder may need to or be preferred to be high in certain other amino acids. For example, a PKU patient is frequently recommended to consume proteins that are particularly high in large neutral amino acids, such as threonine, tryptophan, tyrosine, and leucine. Therefore, a polypeptide suitable for a PKU patient may have an amount greater than what is otherwise considered sufficient for these amino acids. In some embodiments, a polypeptide of the invention comprises at least 50 mg threonine per gram polypeptide (e.g. 50 mg threonine per gram polypeptide, at least 60 mg threonine per gram polypeptide, at least 70 mg threonine per gram polypeptide, at least 80 mg threonine per gram polypeptide, at least 90 mg threonine per gram polypeptide, or at least 100 mg threonine per gram polypeptide); at least 9 mg tryptophan per gram polypeptide (e.g. 9 mg tryptophan per gram polypeptide; at least 15 mg tryptophan per gram polypeptide, at least 20 mg tryptophan per gram polypeptide, at least 25 mg tryptophan per gram polypeptide, at least 30 mg tryptophan per gram polypeptide, at least 35 mg tryptophan per gram polypeptide, at least 40 mg tryptophan per gram polypeptide, at least 45 mg tryptophan per gram polypeptide, or at least 50 mg tryptophan per gram polypeptide); at least 55 mg tyrosine per gram polypeptide (e.g. 55 mg tyrosine per gram polypeptide, at least 65 mg tyrosine per gram polypeptide, at least 75 mg tyrosine per gram polypeptide, at least 85 mg tyrosine per gram polypeptide, at least 95 mg tyrosine per gram polypeptide); and at least 66 mg leucine per gram polypeptide (e.g. 66 mg leucine per gram polypeptide, at least 70 mg leucine per gram polypeptide, at least 80 mg leucine per gram polypeptide, at least 90 mg leucine per gram polypeptide, at least 100 mg leucine per gram polypeptide, or at least 110 mg leucine per gram polypeptide).

In further embodiments, a polypeptide of the invention suitable as a complete protein source for PKU patients comprises at least 60 mg threonine per gram polypeptide; at least 15 mg tryptophan per gram polypeptide; at least 65 mg tyrosine per gram polypeptide; and at least 76 mg leucine per gram polypeptide. In some embodiments, a polypeptide of the invention suitable as a complete protein source for PKU patients comprises at least 70 mg threonine per gram polypeptide; at least 20 mg tryptophan per gram polypeptide; at least 75 mg tyrosine per gram polypeptide; and at least 86 mg leucine per gram polypeptide. In some embodiments, a polypeptide of the invention suitable as a complete protein source for PKU patients comprises at least 80 mg threonine per gram polypeptide; at least 25 mg tryptophan per gram polypeptide; at least 85 mg tyrosine per gram polypeptide; and at least 96 mg leucine per gram polypeptide.

It will be appreciated by one of skill in the art that a strategy similar to the one described above can be used to create a nutritional composition intended for patients suffering metabolic disorders other than PKU. Such diseases include Tyrosinaemia, Maple Syrup Urine Disease, Methylmalonic acidaemia, Homocystinurea, Glutaric aciduria, Isovaleric acidaemia, and Hyperlysinaemia. For example, a nutritional composition suitable as a complete protein source for Tyrosinaemia patients may comprise a polypeptide which has low to no tyrosine and otherwise an AAS ≥0.94.

In some embodiments, a polypeptide of the invention has an AAS≥0.94 without any further constraints on the amount of any amino acid. Such a recombinant polypeptide would be generally useful in a nutritional composition. This nutritional composition may be used as a protein source for someone who does not have a metabolic disorder which restricts protein intake. Such a nutritional composition may be useful to a patient with a reduced appetite or a reduced ability to eat.

In some embodiments, a polypeptide of the invention comprises certain amino acids at amounts above the amount needed to achieve AAS≥0.94. For example, the amino acid leucine is an important factor in stimulating muscle protein synthesis, and more generally branched-chain amino acids (BCAA; includes valine, leucine, and isoleucine) are plentiful in muscle proteins, stimulate muscle growth in the body, and provide energy during exercise. A nutritional composition high in leucine and/or BCAA may be needed and/or desired for groups interested in increasing and/or preserving muscle mass, such as professional athletes (which may be referred to as “sports nutrition”) and the elderly, who tend to lose muscle mass as part of the aging process, or hospitalized people with limited appetite (which may be referred to as “medical nutrition”). Leucine also may play a role in managing blood sugar levels and help control appetite. Therefore, a nutritional composition high in leucine may be needed and/or desired by groups interested in better maintenance of their blood sugar levels and/or weight, such as diabetics, individuals who are pre-diabetic, and individuals struggling with weight control.

In some embodiments, a polypeptide of the invention has an AAS≥0.94 and additionally comprises at least 105 mg leucine per gram of polypeptide, e.g., 105 mg leucine per gram of polypeptide, at least 110 mg leucine per gram of polypeptide, at least 115 mg leucine per gram of polypeptide, at least 120 mg leucine per gram of polypeptide, at least 125 mg leucine per gram of polypeptide, at least 130 mg leucine per gram of polypeptide, at least 135 mg leucine per gram of polypeptide, at least 140 mg leucine per gram of polypeptide, at least 145 mg leucine per gram of polypeptide, at least 150 mg leucine per gram of polypeptide, at least 155 mg leucine per gram of polypeptide, at least 160 mg leucine per gram of polypeptide, at least 170 mg leucine per gram of polypeptide, at least 180 mg leucine per gram of polypeptide, at least 190 mg leucine per gram of polypeptide, at least 200 mg leucine per gram of polypeptide, at least 210 mg leucine per gram of polypeptide, at least 220 mg leucine per gram of polypeptide, at least 230 mg leucine per gram of polypeptide, at least 240 mg leucine per gram of polypeptide, at least 250 mg leucine per gram of polypeptide, at least 275 mg leucine per gram of polypeptide, at least 300 mg leucine per gram of polypeptide, or at least 350 mg leucine per gram of polypeptide.

In some embodiments, a polypeptide of the invention has an AAS≥0.94 and comprises at least 210 mg branched chain amino acids per gram polypeptide, e.g., 210 mg branched chain amino acids per gram polypeptide, at least 215 mg branched chain amino acids per gram polypeptide, at least 220 mg branched chain amino acids per gram polypeptide, at least 225 mg branched chain amino acids per gram polypeptide, at least 230 mg branched chain amino acids per gram polypeptide, at least 235 mg branched chain amino acids per gram polypeptide, at least 240 mg branched chain amino acids per gram polypeptide, at least 245 mg branched chain amino acids per gram polypeptide, at least 250 mg branched chain amino acids per gram polypeptide, at least 255 mg branched chain amino acids per gram polypeptide, at least 260 mg branched chain amino acids per gram polypeptide, at least 265 mg branched chain amino acids per gram polypeptide, at least 270 mg branched chain amino acids per gram polypeptide, at least 275 mg branched chain amino acids per gram polypeptide, at least 280 mg branched chain amino acids per gram polypeptide, at least 285 mg branched chain amino acids per gram polypeptide, at least 290 mg branched chain amino acids per gram polypeptide, at least 300 mg branched chain amino acids per gram polypeptide, at least 310 mg branched chain amino acids per gram polypeptide, at least 320 mg branched chain amino acids per gram polypeptide, at least 330 mg branched chain amino acids per gram polypeptide, at least 340 mg branched chain amino acids per gram polypeptide, at least 350 mg branched chain amino acids per gram polypeptide, at least 360 mg branched chain amino acids per gram polypeptide, at least 370 mg branched chain amino acids per gram polypeptide, at least 380 mg branched chain amino acids per gram polypeptide, at least 390 mg branched chain amino acids per gram polypeptide, at least 400 mg branched chain amino acids per gram polypeptide, at least 425 mg branched chain amino acids per gram polypeptide, at least 450 mg branched chain amino acids per gram polypeptide, at least 475 mg branched chain amino acids per gram polypeptide, or at least 500 mg branched chain amino acids per gram polypeptide. Branched chain amino acids include leucine, isoleucine, and valine.

In further embodiments, a polypeptide of the invention has an AAS≥0.94 and comprises at least 105 mg leucine per gram polypeptide and further comprises at least 210 mg branched chain amino acids per gram polypeptide.

Polypeptides of the invention may have their amino acid composition optimized to provide ideal amino acid compositions for nutritional compositions suitable for the protein and amino acid needs of any group. For example, higher amounts of protein and enrichment of certain amino acids may also be required during pregnancy. In some embodiments, a nutritional composition of the invention comprises a recombinant polypeptide of the invention with an amino acid composition optimized for pregnant women. It is recognized that amino acid needs may change throughout pregnancy. Polypeptides of the invention may have their amino acid composition optimized to provide ideal amino acid compositions for nutritional compositions suitable for each stage of pregnancy.

### Variants

Polypeptides of the invention include variants of any one of SEQ ID NOs: 3 to 101, where the variant comprises an amino acid sequence with at least 60%, at least 65%, at least 70%, at least 75%, at least 80%, at least 85%, at least 90%, at least 95%, or 100% sequence identity to the amino acid sequence of SEQ ID NO: 3 to 101 or a truncate thereof. The variants can be prepared using any mutagenesis procedure known in the art, such as site-directed mutagenesis, synthetic gene construction, semi-synthetic gene construction, random mutagenesis, shuffling, etc.

A variant has a different amino acid composition compared to its parent polypeptide, which may be optimized to be a complete protein source for daily consumption for any one of a number of different amino acid requirements, as described above. In some embodiments, the variant has an AAS≥0.94 but has a different amino acid composition compared to any one of SEQ ID NOs: 3 to 101. Such a variant may be useful in a nutritional composition.

In some embodiments, variants may have improved properties, for example improved stability under storage conditions or improved thermostability compared to the parent polypeptide.

In some embodiments, the variant has improved ease of purification compared to the parent polypeptide. In some embodiments, the improved ease of purification may be a greater yield when following the same purification steps as the parent polypeptide. In some embodiments, the improved ease of purification may require fewer steps. In some embodiments, the improved ease of purification may be the purification process is less resource intensive, meaning that it may use less energy, may take less time, or may use regents or materials that cost less.

In an embodiment, the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of any one of SEQ ID NOs: 3 to 101.

In one aspect, the number of alterations in the variants of the present invention is 1-20, e.g., 1-10 or 1-5, such as 1, 2, 3, 4, 5, 6, 7, 8, 9 or 10 alterations.

In another aspect, a variant comprises a substitution, an insertion or a deletion at one or more positions of the amino acid sequence of any one of SEQ ID NOs: 3 to 101. In another aspect, a variant comprises a substitution, an insertion or a deletion at two or more positions of the amino acid sequence of any one of SEQ ID NOs: 3 to 101. In another aspect, a variant comprises a substitution, an insertion or a deletion at three or more positions of the amino acid sequence of any one of SEQ ID NOs: 3 to 101. In another aspect, a variant comprises a substitution, an insertion or a deletion at four or more positions of the amino acid sequence of any one of SEQ ID NOs: 3 to 101.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a polyhistidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, change the pH optimum, improve purification yield, and the like.

The polypeptides of the invention are desirable for their amino acid composition and for their ease of purification. Amino acids in a polypeptide that are critical for stability (which may affect ease of purification) or for ease of purification itself can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for stability or ease of purification to identify amino acid residues that are critical. See also, Hilton et al., 1996, J. Biol. Chem. 271:4699-4708. Amino acid residues critical for stability can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of amino acids critical for stability or for ease of purification can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify amino acids critical for stability or for ease of purification. See, for example, Jumper et al., 2021, “Highly accurate protein structure prediction with AlphaFold”, Nature 596:583-589.

The variants may consist of 500 to 450, 450 to 400, 400 to 350, 350 to 300, 300 to 250, 250 to 200, 200 to 150, 150 to 100, or 100 to 50 amino acids of any one of SEQ ID NOs: 3 to 101.

A polypeptide of the invention may be a fusion polypeptide comprising a variant of the invention.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

### Promoters

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenbock, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177:3465-3471).

Examples of mRNA stabilizer regions for fungal cells are described in Geisberg et al., 2014, Cell 156 (4): 812-824, and in Morozov et al., 2006, Eukaryotic Cell 5 (11): 1838-1846.

### Leader Sequences

The control sequence may also be a leader, a nontranslated region of an mRNA that is important for translation by the host cell. The leader is operably linked to the 5′-terminus of the polynucleotide encoding the variant. Any leader that is functional in the host cell may be used.

Preferred leaders for filamentous fungal host cells may be obtained from the genes for Aspergillus oryzae TAKA amylase and Aspergillus nidulans triose phosphate isomerase.

### Polyadenylation Sequences

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

### Signal Peptides

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40:949-955

### Propeptides

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained for example from the genes for Bacillus subtilis alkaline protease (aprE), Bacillus subtilis neutral protease (nprT), Myceliophthora thermophila laccase (WO 95/33836), Rhizomucor miehei aspartic proteinase, or Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

### Regulatory Sequences

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52:7-23, as well in Balleza et al., 2009, FEMS Microbiol. Rev. 33 (1): 133-151.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a variant of the present invention.

A construct or vector comprising a polynucleotide is introduced into a host cell so that the construct or vector is maintained as a chromosomal integrant or as a self-replicating extrachromosomal vector as described earlier. The choice of a host cell will to a large extent depend upon the gene encoding the variant and its source. The recombinant host cell may comprise a single copy, or at least two copies, e.g., three, four, five, or more copies of the polynucleotide of the present invention.

The host cell may be any cell useful in the recombinant production of a variant of the invention, e.g., a prokaryotic cell or a fungal cell.

The host cell may be any microbial cell useful in the recombinant production of a polypeptide of the present invention, e.g., a prokaryotic cell or a fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16:168 and procedures described in EP 238023, Yelton et al., 1984, Proc. Natl. Acad. Sci. USA 81:1470-1474, Christensen et al., 1988, Bio/Technology 6:1419-1422, and Lubertozzi and Keasling, 2009, Biotechn. Advances 27:53-75. However, any method known in the art for introducing DNA into a fungal host cell can be used, and the DNA can be introduced as linearized or as circular polynucleotide.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

The host cell may be a prokaryotic cell. The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168:111-115), competent cell transformation (see, e.g., Young and Spizizen, 1961, J. Bacteriol. 81:823-829, or Dubnau and Davidoff-Abelson, 1971, J. Mol. Biol. 56:209-221), electroporation (see, e.g., Shigekawa and Dower, 1988, Biotechniques 6:742-751), or conjugation (see, e.g., Koehler and Thorne, 1987, J. Bacteriol. 169:5271-5278). The introduction of DNA into an E. coli cell may be effected by protoplast transformation (see, e.g., Hanahan, 1983, J. Mol. Biol. 166:557-580) or electroporation (see, e.g., Dower et al., 1988, Nucleic Acids Res. 16:6127-6145). The introduction of DNA into a Streptomyces cell may be effected by protoplast transformation, electroporation (see, e.g., Gong et al., 2004, Folia Microbiol. (Praha) 49:399-405), conjugation (see, e.g., Mazodier et al., 1989, J. Bacteriol. 171:3583-3585), or transduction (see, e.g., Burke et al., 2001, Proc. Natl. Acad. Sci. USA 98:6289-6294). The introduction of DNA into a Pseudomonas cell may be effected by electroporation (see, e.g., Choi et al., 2006, J. Microbiol. Methods 64:391-397), or conjugation (see, e.g., Pinedo and Smets, 2005, Appl. Environ. Microbiol. 71:51-57). The introduction of DNA into a Streptococcus cell may be effected by natural competence (see, e.g., Perry and Kuramitsu, 1981, Infect. Immun. 32:1295-1297), protoplast transformation (see, e.g., Catt and Jollick, 1991, Microbios 68:189-207), electroporation (see, e.g., Buckley et al., 1999, Appl. Environ. Microbiol. 65:3800-3804) or conjugation (see, e.g., Clewell, 1981, Microbiol. Rev. 45:409-436). However, any method known in the art for introducing DNA into a host cell can be used.

In some embodiments, the host cell is isolated. In some embodiments, the host cell is purified.

### Methods of Production

The present invention also relates to methods of producing a polypeptide of the present invention, comprising (a) cultivating a recombinant host cell of the present invention under conditions conducive for production of the polypeptide; and (b) recovering the polypeptide.

The host cell is cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an enzyme assay determining the relative or actual amount of the polypeptide.

The polypeptide may be recovered from the medium using methods known in the art, including, but not limited to, collection, centrifugation, filtration, extraction, spray-drying, evaporation, or precipitation. In one aspect, the whole fermentation broth is recovered. In another aspect, a cell-free fermentation broth comprising the polypeptide is recovered.

The polypeptide may be purified by a variety of procedures known in the art to obtain substantially pure polypeptides and/or fragments (see, e.g., Wingfield, 2015, Current Protocols in Protein Science; 80 (1): 6.1.1-6.1.35; Labrou, 2014, Protein Downstream Processing, 1129:3-10).

In an alternative aspect, the polypeptide is not recovered, but for example a host cell of the present invention expressing the variant is used as a source of the variant.

### Nutritional Compositions

The nutritional composition of the invention comprises polypeptides disclosed herein. To be suitable for a nutritional composition, polypeptides of the invention need to be soluble and remain intact in a range of pH and temperatures.

A nutritional composition typically comprises a pH between 3 and 8. A polypeptide of the invention needs to remain soluble at the pH of the nutritional composition which it comprises. In some embodiments, a polypeptide of the invention remains soluble at a pH of at least about pH 2.0, pH 2.5, pH 3.0, pH 3.5, pH 4.0, pH 4.5, pH 5.0, pH 5.5, pH 6.0, pH 6.5, pH 7.0, pH 7.5, or pH 8.0. In some embodiments, a polypeptide of the invention remains soluble at a pH of about pH 3.0, pH 3.5, pH 4.0, pH 4.5, pH 5.0, pH 5.5, pH 6.0, pH 6.5, pH 7.0, pH 7.5, or pH 8.0. In some embodiments, the polypeptide of the invention remains soluble at the given pH both before, during, and after a heat treatment.

A nutritional composition may be heat treated for pasteurization or sterilization. The heat treatment is intended to make the nutritional composition safe for consumption by killing harmful microorganisms, thereby reducing the risk of food poisoning. The heat-treatment also increases shelf-life or shelf-stability of the nutritional composition, as it eliminates microorganisms in the beverage which may degrade the beverage, either by causing it to spoil or by decreasing the quality of the beverage.

In some embodiments, the heat treatment is 85-95° C. for 10 seconds to 30 minutes. In some embodiments, the heat treatment is High Temperature Short Time (HTST) pasteurization. For this, the composition is heated to a temperature between 71.5-74° C. for 15-30 seconds, or is heated to a temperature between 74-76° C. for 15-20 seconds. Following heat treatment, the nutritional composition may then be rapidly cooled to 4-5.5° C. In some embodiments, the acidic probiotic beverage is heat-treated by an Ultra High Temperature (UHT) treatment. The UHT treatment may be direct or indirect. In some embodiments, the UHT treatment is 135-154° C. for 1-10 seconds. In some embodiments, the nutritional composition is heat-treated by ultra pasteurization. For this, the composition is heated to a temperature between 70-75° C. for 20-30 minutes.

In some embodiments, a polypeptide of the invention possesses qualities, such as for example a high melting temperature, that indicate it will remain intact throughout a heat treatment.

The nutritional composition may comprise additional ingredients. In some embodiments, the nutritional composition of the invention comprises a fat source. The fat source may be any suitable fat or fat mixture. In some embodiments, the fat source is a vegetable fat. The vegetable fat may be soy oil, palm oil, coconut oil, safflower oil, sunflower oil, corn oil, canola oil, lecithins, or any suitable vegetable fat. In some embodiments, the fat source is an animal source, such as a milk fat. In some embodiments, the fat source is derived from a vegetable source, such as fractionated vegetable oil. In some embodiments, the fat source provides about 20% to 70% of the energy in the nutritional composition. In further embodiments, the fat source provides about 25% to 60% of the energy in the nutritional composition.

In some embodiments, the nutritional composition comprises a carbohydrate source. The carbohydrate source may be any suitable carbohydrate, including sucrose, lactose, glucose, fructose, corn syrup, corn syrup solids, and/or maltodextrins. In some embodiments, the carbohydrate source provides about 20% to 70% of the energy in the nutritional composition. In further embodiments, the carbohydrate source provides about 30% to 60% of the energy in the nutritional composition.

In some embodiments, the nutritional composition comprises vitamins and/or minerals needed in the diet. Vitamins include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, choline or any combination thereof. Minerals include boron, calcium, chromium, copper, iodine, iron, magnesium, manganese, molybdenum, nickel, phosphorus, potassium, selenium, silicon, tin, vanadium, zinc, or any combination thereof.

In some embodiments, the nutritional composition comprises additional components which may be beneficial to gut health and/or health overall. These additional components include omega-3 fatty acids such as a-linolenic acid, stearidonic acid, docosahexaenoic acid, eicosapentaenoic acid; phytonutrients such as carotenoids, plant sterols, quercetin, curcumin, limonin, compounds such as a-ketoglutarate, L-carnitine; or antioxidants such as astaxanthin, coenzyme Q10, flavonoids, glutathione, hesperidin, lactowolfberry, lignan, lutein, lycopene, polyphenols, selenium, or zeaxanthin, or any combination thereof.

In some embodiments, the nutritional composition comprises prebiotics. The prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or any combination thereof.

In some embodiments, the nutritional composition comprises probiotics. The probiotics include microorganisms such as Aerococcus, Aspergillus, Bacteroides, Bacillus, Bifidobacterium, Brevibacillus, Candida, Clostridium, Debaromyces, Enterococcus, Fusobacterium, Lactobacillus, Lactococcus, Leuconostoc, Melissococcus, Micrococcus, Mucor, Oenococcus, Paenibacillus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or any combination thereof.

In some embodiments, the nutritional composition may also include any number of optional additional ingredients, including conventional food additives (synthetic or natural), for example one or more acidulants, additional thickeners, buffers or agents for pH adjustment, chelating agents, colorants, emulsifiers, excipient, flavorants, minerals, osmotic agents, acceptable carriers, preservatives, stabilizers, sugars, sweeteners, texturizers, minerals, and/or vitamins. The optional ingredients can be added in any suitable amount.

The nutritional composition of the invention may be prepared by mixing together, in powdered form, the recombinant polypeptide, optionally with fat or carbohydrate sources or other additional components. The nutritional composition may also be prepared by adding the ingredients together in a liquid form and then spray dry them to a powder. Suitable dosages forms for the nutritional composition of the invention include tablets, dispersible powders, granules, capsules, liquid, suspensions, and syrups.

Inert diluents and carriers for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate and stearic acid. Tablets may be uncoated or may be coated by known techniques to delay disintegration and absorption. Inert diluents and carriers which may be used in capsules include, for example, calcium carbonate, calcium phosphate, and kaolin. Suspensions, liquids, and syrups may contain conventional excipients, for example, methyl cellulose, tragacanth, sodium alginate; wetting agents, such as lecithin and polyoxyethylene stearate; and preservatives, such as ethyl-p-hydroxybenzoate.

The nutritional composition of the invention may be added to food ordinarily consumed by a patient. In some embodiments, the nutritional composition may be suitable as a complete protein source. In some embodiment, the nutritional composition may be suitable for an individual with increased medical needs and/or long term care, including for example the elderly, pregnant women, cancer patients, and individuals with long-term diseases such as diabetes. In some embodiments, the nutritional composition may be suitable as a complete protein source for a patient who has a metabolic disorder. In further embodiments, the nutritional composition may be suitable as a complete protein source for a patient who has PKU and/or HPA.

### Preferred Embodiments

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered embodiments.

1. A nutritional composition comprising a recombinant polypeptide comprising an amino acid sequence with at least 60%, at least 65%, at least 70%, at least 75%, at least 80%, at least 85%, at least 90%, at least 95%, or 100% sequence identity to the amino acid sequence of SEQ ID NO: 3-101, or a truncate thereof.

2. The nutritional composition of claim 1, wherein the recombinant polypeptide comprises an amino acid sequence with at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3-101, or a truncate thereof.

3. The nutritional composition of claim 1 or claim 2, wherein the recombinant polypeptide comprises the amino acid sequence of any one of SEQ ID NOs: 3-101.

4. The nutritional composition of any of the preceding claims, wherein the recombinant polypeptide comprises an amino acid sequence with at least 60%, at least 65%, at least 70%, at least 75%, at least 80%, at least 85%, at least 90%, at least 95%, at least 96%, at least 97%, at least 98%, at least 99% sequence identity, or at least 100% sequence identity, to the amino acid sequence of SEQ ID NO: 3-11, 45-48, 50, or 51 or a truncate thereof.

5. The nutritional composition of any one of the proceeding claims, wherein the recombinant polypeptide comprises an amino acid sequence with at least 60%, at least 65%, at least 70%, at least 75%, at least 80%, at least 85%, at least 90%, at least 95%, or 100% sequence identity to a truncate of amino acid sequence of SEQ ID NO: 50, wherein the truncate possesses an N-terminal truncate, C-terminal truncate, and/or an internal truncation.

6. The nutritional composition of claim 5, wherein the recombinant polypeptide comprises an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 93%, at least 94%, at least 95%, at least 96%, at least 97%, at least 98%, at least 99%, or 100% sequence identity to the amino acid sequence of SEQ ID NO: 50, wherein the truncate has an N-terminal truncation of at least 1 amino acid and up to 350 amino acids relative to the amino acid sequence of SEQ ID NO: 50.

7. The nutritional composition of claim 5, wherein the recombinant polypeptide comprises an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the of amino acid sequence of SEQ ID NO: 50, wherein the truncate has a C-terminal truncation of at least 1 amino acid and up to 350 amino acids relative to the amino acid sequence of SEQ ID NO: 50.

8. The nutritional composition of claim 5, wherein the recombinant polypeptide comprises an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the of amino acid sequence of SEQ ID NO: 50, wherein the truncate has an internal truncation of at least 1 amino acid and up to 350 amino acids relative to the amino acid sequence of SEQ ID NO: 50.

9. The nutritional composition of claim 5, wherein the recombinant polypeptide comprises an amino acid sequence with at least 60%, at least 65%, at least 70%, at least 75%, at least 80%, at least 85%, at least 90%, at least 91%, at least 92%, at least 93%, at least 93%, at least 94%, at least 95%, at least 96%, at least 97%, at least 98%, at least 99% sequence identity, or at least 100% sequence identity, to the amino acid sequence of any one of SEQ ID NOs: 4-48, 52, 53, 58, 61, 62, 63, 66, 67, and 70-75.

10. The nutritional composition of any one of the proceeding claims, wherein the recombinant polypeptide has an Amino Acid Score (AAS) greater than or equal to 0.94.

11. The nutritional composition of any one of the proceeding claims, wherein the recombinant polypeptide comprises:
a) at least 50 mg threonine per gram polypeptide;
b) at least 9 mg tryptophan per gram polypeptide;
c) at least 55 mg tyrosine per gram polypeptide; and
d) at least 66 mg leucine per gram polypeptide.

12. The nutritional composition of any one of the proceeding claims, wherein the composition does not comprise added free amino acids.

13. The nutritional composition of any one of the proceeding claims, further comprising vitamins and minerals.

14. The nutritional composition of any one of the proceeding claims, further comprising a carbohydrate source and/or a fat source.

15. The nutritional composition of any one of the proceeding claims, wherein the composition is a tablet, dispersible powder, granule, capsule, liquid, suspension, or syrup.

16. The nutritional composition of any one of the proceeding claims, wherein the recombinant polypeptide comprises a nutritionally complete amino acid profile sufficient for patients with metabolic disorders.

17. The nutritional composition of any one of the proceeding claims, wherein the recombinant polypeptide comprises a nutritionally complete amino acid profile sufficient for patients with Phenylketonuria, Hyperphenylalaninemia, Tyrosinaemia, Maple Syrup Urine Disease, Methylmalonic acidaemia, Homocystinurea, Glutaric aciduria, Isovaleric acidaemia, and/or Hyperlysinaemia.

18. The nutritional composition of claim 16 or 17, wherein the recombinant polypeptide does not comprise the amino acid(s) which are harmful to the patient of the metabolic disorder.

19. The nutritional composition of any one of claims 16-18, wherein the recombinant polypeptide has an AAS equal to or greater than 0.94, aside from amino acid(s) which are harmful to the patient of the metabolic disorder.

20. The nutritional composition of any one of claims 16-19, wherein the recombinant polypeptide comprises no phenylalanine residues.

21. The nutritional composition of claim 20, wherein the recombinant polypeptide has an AAS equal to or greater than 0.94, aside from phenylalanine.

22. The nutritional composition of any one of claims 16-21, wherein the recombinant polypeptide comprises an amino acid sequence with at least 90%, at least 91%, at least 92%, at least 93%, at least 93%, at least 94%, at least 95%, at least 96%, at least 97%, at least 98%, at least 99%, or 100% sequence identity to the amino acid sequence of SEQ ID NO: 3-48.

23. The nutritional composition of any one of claims 1-15, wherein the recombinant polypeptide has an AAS equal to or greater than 0.94, and further where the recombinant polypeptide comprises at least 105 mg leucine per gram polypeptide.

24. The nutritional composition of any one of claim 1-15 or 23, wherein the recombinant polypeptide has an AAS equal to or greater than 0.94, and further where the recombinant polypeptide comprises at least 210 mg branched chain amino acids per gram polypeptide.

25. The nutritional composition of any one of claim 1-15, 23, or 24, wherein the recombinant polypeptide comprises the amino acid sequence of SEQ ID NOs: 78-101.

26. A recombinant polypeptide comprising an amino acid sequence with at least 60%, at least 65%, at least 70%, at least 75%, at least 80%, at least 85%, at least 90%, at least 95%, or 100% sequence identity to the amino acid sequence of SEQ ID NO: 3-101 or a truncate thereof, and further comprises a substitution at a position corresponding to position E334 and/or E426 of SEQ ID NO: 49.

27. The recombinant polypeptide of claim 26, comprising an amino acid sequence with at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 3-101 or a truncate thereof, and further comprises a substitution at a position corresponding to position E334 and/or E426 of SEQ ID NO: 49.

28. The recombinant polypeptide of claim 26 or claim 27, wherein the substitution at the position corresponding to position E334 of SEQ ID NO: 49 is to an A, I, G, L, M, Q, or V.

29. The recombinant polypeptide of claim 26 or claim 27, wherein the substitution at the position corresponding to position E426 of SEQ ID NO: 49 is to an A, I, G, L, M, Q, or V.

30. The recombinant polypeptide of any of claim 26-29, wherein the recombinant polypeptide comprises an amino acid sequence with at least 60%, at least 65%, at least 70%, at least 75%, at least 80%, at least 85%, at least 90%, at least 95%, at least 96%, at least 97%, at least 98%, at least 99% sequence identity, or at least 100% sequence identity, to the amino acid sequence of SEQ ID NO: 3-11,45-48, 50, 51, or a truncate thereof.

31. The recombinant polypeptide of any one of claims 26-30, wherein the recombinant polypeptide comprises an amino acid sequence with at least 60%, at least 65%, at least 70%, at least 75%, at least 80%, at least 85%, at least 90%, at least 95%, or 100% sequence identity to a truncate of amino acid sequence of SEQ ID NO: 50, wherein the truncate possesses an N-terminal truncate, C-terminal truncate, and/or an internal truncation.

32. The recombinant polypeptide of claim 31, wherein the recombinant polypeptide comprises an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 93%, at least 94%, at least 95%, at least 96%, at least 97%, at least 98%, at least 99%, or 100% sequence identity to the amino acid sequence of SEQ ID NO: 50, wherein the truncate has an N-terminal truncation of at least 1 amino acid and up to 350 amino acids relative to the amino acid sequence of SEQ ID NO: 50.

33. The recombinant polypeptide of claim 31, wherein the recombinant polypeptide comprises an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the of amino acid sequence of SEQ ID NO: 50, wherein the truncate has a C-terminal truncation of at least 1 amino acid and up to 350 amino acids relative to the amino acid sequence of SEQ ID NO: 50.

34. The recombinant polypeptide of claim 31, wherein the recombinant polypeptide comprises an amino acid sequence with at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the of amino acid sequence of SEQ ID NO: 50, wherein the truncate has an internal truncation of at least 1 amino acid and up to 350 amino acids relative to the amino acid sequence of SEQ ID NO: 50.

35. The recombinant polypeptide of claim 31, wherein the recombinant polypeptide comprises an amino acid sequence with at least 60%, at least 65%, at least 70%, at least 75%, at least 80%, at least 85%, at least 90%, at least 91%, at least 92%, at least 93%, at least 93%, at least 94%, at least 95%, at least 96%, at least 97%, at least 98%, at least 99% sequence identity, or at least 100% sequence identity, to the amino acid sequence of any one of SEQ ID NOs: 4-48, 52, 53, 58, 61, 62, 63, 66, 67, and 70-75.

36. The recombinant polypeptide of any one of claims 26-35, wherein the recombinant polypeptide has an Amino Acid Score (AAS) greater than or equal to 0.94.

37. The recombinant polypeptide any one of claims 26-36, wherein the recombinant polypeptide comprises:
a) at least 50 mg threonine per gram polypeptide;
b) at least 9 mg tryptophan per gram polypeptide;
c) at least 55 mg tyrosine per gram polypeptide; and
d) at least 66 mg leucine per gram polypeptide.

38. The recombinant polypeptide of any one of claim 26-34 or 37, wherein the recombinant polypeptide comprises no phenylalanine residues.

39. The recombinant polypeptide of claim 38, wherein the recombinant polypeptide has an AAS equal to or greater than 0.94, aside from phenylalanine.

40. The recombinant polypeptide of claim 38 or claim 39, wherein the recombinant polypeptide comprises any one of the amino acid sequence of SEQ ID NOs: 3-11 and 45-48.

41. The recombinant polypeptide of any one of claims 26-36, wherein the recombinant polypeptide has an AAS equal to or greater than 0.94, and further where the recombinant polypeptide comprises at least 105 mg leucine per gram polypeptide.

42. The recombinant polypeptide of any one of claim 26-36 or 41, wherein the recombinant polypeptide has an AAS equal to or greater than 0.94, and further where the recombinant polypeptide comprises at least 210 mg branched chain amino acids per gram polypeptide.

43. The recombinant polypeptide of any one of claim 26-36, 41, or 42, wherein the recombinant polypeptide comprises the amino acid sequence of SEQ ID NOs: 78-101.

44. The use of a recombinant polypeptide of any of claims 26-43 in a nutritional composition.

45. The use of a recombinant polypeptide of any of claims 26-43 in a nutritional composition for patients with metabolic disorders.

46. An isolated polynucleotide encoding the polypeptide of any one of claims 26-43.

47. A nucleic acid construct or expression vector comprising the polynucleotide of claim 46.

48. A recombinant host cell transformed with the polynucleotide of claim 46.

49. A method of producing a polypeptide comprising cultivating the recombinant host cell of claim 48 under conditions suitable for expression of the polypeptide and recovering the polypeptide.

Various references are cited herein, the disclosures of which are incorporated by reference in their entireties. The present invention is further described by the following examples which should not be construed as limiting the scope of the invention.

### EXAMPLES

### Example 1: Production of a Recombinant Polypeptide for a Nutritional Composition for General Nutrition

A recombinant polypeptide was identified which was found to express at very high levels when expressed recombinantly in A. niger, A. oryzae and Bacillus licheniformis. Unexpectedly, this polypeptide is suitable in a nutritional composition, as it possesses an amino acid composition which is close to a desirable AAS and also does not contain either toxic domains or amino acid motifs known to play a role in protein allergens. Although the polypeptide has mannanase activity (described in WO2021/152123, incorporated by reference herein), an inactive variant can be produced by introducing a substitution at position 334 and/or 426 (positions relative to SEQ ID NO: 49).

The combination of a polypeptide which possesses a desirable amino acid composition and no known toxic or allergenic domains, and also expresses highly with no detectable nonspecific carry-over from purification is unpredictable and highly desirable for a recombinant polypeptide for a nutritional composition. The high AAS of this polypeptide also makes this polypeptide suitable as nutritious protein in categories beyond general nutrition, such as sport beverages or as medical nutrition for elderly and hospitalized people with limited appetite. Therefore, it was determined if inactive variant polypeptides still had the same useful qualities for ease of purification and purity of sample.

The polypeptide encoded by SEQ ID NO: 49 is an active mannanase. The polypeptide encoded by SEQ ID NO: 50 is an inactivated mannanase. A nucleic acid construct encoding either SEQ ID NO: 49 or 50 was introduced into strains of the filamentous fungi A. niger. These recombinant strains were fermented using standard protocols (3-4 days, 30° C.), and the recombinant polypeptides were purified by chromatography using standard ion-exchange techniques. Expression and purification of the recombinant polypeptides were analyzed using SDS-PAGE as well as MS proteomics after tryptic digest. The recombinant polypeptides of both SEQ ID NOs: 49 and 50 were found to express at a high level.

Yield and purity data from 2 different fermentations of which produced the polypeptide of SEQ ID NO: 49 and a single fermentation which produced the polypeptide of SEQ ID NO: 50 are provided in Table 1. SDS-PAGE analysis positively identified the protein of interest, and densiometric scanning of the SDS-PAGE gel enabled a relative quantification of the purified protein. Results indicate robust yield and excellent purity.

TABLE 1
Yield and Purity of recombinant polypeptides

| | Fermentation | Purified |
|---|---|---|
| Fermentation | Supernatant | sample |
| SEQ ID NO: 49, fermentation 1 | 87%-93% | 100% |
| SEQ ID NO: 49, fermentation 2 | 95% | 100% |
| SEQ ID NO: 50 | 94% | 100% |

In addition to the purity estimated by SDS-PAGE, the expression of the protein variant relative to host strain proteins was estimated using LC-MS/MS. Tryptic digests were prepared by a Filter-Aided Sample Preparation (FASP) method. Briefly, following tryptic digestion the extracted peptides were analyzed on a nanoLC-MS/MS system: Evosep One (Evosep, Denmark)/timsTOF Pro (Bruker Daltonik, Massachusetts, USA). For protein identification the data were searched against available internal and public databases using the Mascot search engine (Matrix Science, London, UK) using Genedata Expressionist software with a 1% False Discovery Rate cutoff. Relative protein concentrations were calculated by label free quantification from peptide volumes in Genedata Expressionist. Results are shown in Table 2.

TABLE 2
LC-MS/MS data for yield and purity of recombinant polypeptides

| | Fermentation | Purified |
|---|---|---|
| Fermentation | Supernatant | sample |
| SEQ ID NO: 49, fermentation 1 | 94% | 97% |
| SEQ ID NO: 49, fermentation 2 | 93% | 98% |
| SEQ ID NO: 50 | 87% | 98% |

The LC-MS/MS data in Table 2 confirms the conclusion from the SDS-PAGE analysis on the high ratio of target protein to host-strain proteins. This indicates the purification process successfully removed cell debris and other non-proteinaceous components from the fermentation that can impact functionality in food products as well as the flavor profile of the final product.

### Example 2: Production of a Recombinant Polypeptide for a Nutritional Composition for Metabolic Disorders

As shown in Example 1, an inactive variant of the mannanase can be produced a high expression and purity levels. Therefore, further mutations were introduced to test if other variants of the polypeptide would still have the ease of purification and high yield and purity.

The polypeptides of SEQ ID NO: 49 and 50 have a relatively low phenylalanine composition. A variant polypeptide was produced in which the phenylalanines were substituted with other amino acids. Substitutions were also introduced to balance the amino acid composition so that the polypeptide has an AAS ≥0.94, other than the absence of phenylalanine. An example of nucleic acid sequence of an inactive, Phe-free recombinant polypeptide with an AAS ≥0.94 is SEQ ID NO: 1. The corresponding amino acid sequence of the recombinant polypeptide is SEQ ID NO: 2, and the corresponding amino acid sequence of the mature polypeptide, where the signal peptide is removed, is SEQ ID NO: 3.

A nucleic acid construct comprising a nucleotide sequence encoding for SEQ ID NO: 3 was introduced into a strain of the filamentous fungi A. oryzae. This recombinant strain was fermented using standard protocols (3-4 days, 30° C.) and then purified by chromatography using standard ion exchange techniques. Expression and purification of the recombinant polypeptide was analyzed using SDS-PAGE. The recombinant polypeptide of SEQ ID NO: 3 was found to express at a high level.

Data from fermentation and purification of the recombinant polypeptide of SEQ ID NO: 3 using 4 different media are shown in Table 3. SDS-PAGE analysis positively identified the protein of interest, and densiometric scanning of the SDS-PAGE gel enabled a relative quantification of the purified protein. Results indicate robust yield and excellent purity in a range of media.

TABLE 3
Yield and Purity using different fermentation media

| | Fermentation Supernatant | Purified sample |
|---|---|---|
| Media 1 | More than 95% | 100% |
| Media 2 | More than 95% | 100% |
| Media 3 | More than 95% | 100% |
| Media 4 | More than 95% | 100% |

The phenylalanine levels in the purified samples was estimated by total amino acid analysis. Although the polypeptide of SEQ ID NO: 3 contained no phenylalanines, it is possible that phenylalanine could be present in the purified sample as a result of background phenylalanine carrying over from the expression host, such as in cellular debris. The amount of residual phenylalanine present in the purified protein was determined using a standard amino acid analysis method, where amino acids were derivatized using AccQ-Tag Ultra Reagent (Waters Corp., Milford, MA) and separated using reversed-phase UPLC (UPLC®, Waters Corp., Milford, MA). The derivatives were quantitated based on UV absorbance. The detection limit for phenylalanine using this method is 2 μmol. No phenylalanine was detected, indicating that very little to no phenylalanine was present in the purified sample.

### Example 3: In Vitro Digestibility

The digestibility of the purified recombinant polypeptides described in Examples 1 and 2 was determined using the method published by Minekus et al. (“A standardised static in vitro digestion method suitable for food—an international consensus”, Food Funct, 5:1113-1124, 2014). The treated samples were then analyzed by SDS-PAGE. Results are shown in Table 4.

TABLE 4
In vitro digestibility

| Polypeptide | Oral | Gastric | Intestinal |
|---|---|---|---|
| SEQ ID NO: 3 | − | +++ | +++ |
| SEQ ID NO: 49 | − | +++ | +++ |
| SEQ ID NO: 50 | − | +++ | +++ |

None of the variants were degraded in the simulated oral phase while all variants showed extensive degradation in the simulated gastric phase, with only a few peptide fragments detectable by SDS-PAGE. The few fragments detectable after the simulated gastric phase were fully degraded in the simulated intestinal phase. In conclusion, the in vitro digestibility assay points to complete digestibility of these molecules and thus high bioavailability of the individual amino acids.

### Example 4: Heat Stability and Solubility

Use of polypeptides for food applications requires more than a high nutritional value to succeed. Functionalities like heat stability and solubility are important to enable good quality finished nutritional compositions, such as powders, beverages, and processed foods such as bars.

Heat stability of purified polypeptides comprising the amino acid sequences of SEQ ID NO: 49 or 50 were investigated by nano-Differential scanning fluorimetry (nanoDSF) using equipment from NanoTemper Technologies (Munich, Germany). nanoDSF is a biophysical characterization technique used for assessing the conformational stability of a biological sample. It uses the intrinsic fluorescence of a protein to monitor how it responds to stress inputs such as temperature or chaotropes. This information is used to determine conformational stability of the protein, and to rank candidates or buffer formulations based on their impact on this stability.

The heat stability of a protein is characterized by the on-set of unfolding (Tₒₙ) and the melting temperature where 50% of the protein is un-folded (Tₘ). Results are shown in Table 5.

TABLE 5
Heat stability

| Polypeptide | Tₒₙ (° C.) | Tₘ (° C.) |
|---|---|---|
| SEQ ID NO: 49 | 73.6 | 78.7 |
| SEQ ID NO: 50 | 66.2 | 78.7 |

The high melting temperature of the purified polypeptides indicates that these polypeptides can remain intact throughout a heat treatment, such as a High-Temperature Short Time (HTST) pasteurization or similar heat-treatments for pasteurization or sterilization typically used in the food industry.

Solubility is another key factor in determining suitability for a polypeptide in the production of nutritional compositions. For the polypeptides of SEQ ID NO: 49 and 50, the pH dependence of solubility was determined with and without a short heat treatment. Solubility of solutions comprising the polypeptide of SEQ ID NO: 49 or 50 (10 mg/mL) in a range of pH from 3 to 8 were determined before and after a heat-treatment of 3 min at 95° C. Percent solubility was determined by measuring absorbance at 280 nm before and after centrifugation (to remove precipitate) and calculating the ratio. Percent solubility was determined before and after heat-treatment. Results are shown in Table 6.

TABLE 6
Solubility at a range of pH with heat treatment

| | Pre-heat treatment | Heat-treated | Pre-heat treatment | Heat-treated |
|---|---|---|---|---|
| pH | SEQ ID NO: 50 | SEQ ID NO: 50 | SEQ ID NO: 49 | SEQ ID NO: 49 |
| 3 | 102% | 101% | 99% | 99% |
| 4 | 107% | 103% | 100% | 2% |
| 5 | 8% | 1% | 16% | 0% |
| 6 | 92% | 5% | 101% | 2% |
| 7 | 100% | 107% | 101% | 106% |
| 8 | 99% | 113% | 98% | 105% |

Before heat-treatment, the samples showed protein solubilities close to 100% except in pH 5, which is close to the pl of the polypeptide. In water at neutral pH, the polypeptides encoding the amino acid sequences of SEQ ID NO: 49 and 50 showed a high solubility (more than 10% w/w). After heat treatment, solubility decreased for both polypeptides. However, the inactive variant polypeptide comprising SEQ ID NO: 50 demonstrated a more favorable solubility profile after heat treatment, indicating that inactive variants are good candidates for use in nutritional compositions.

The heat stability of a number of variants is shown in Table 7. Heat stability was determined by nanoDSF as described above. The variants have a range of unfolding temperatures (Tm) of more than 20° C., thus indicating the broad range in which variants of the recombinant polypeptide may be produced for specific applications with various requirements for heat stability.

TABLE 7
Heat stability of polypeptide variants

| SEQ ID NO. | Tₒₙ (° C.) | Tₘ (° C.) |
|---|---|---|
| 54 | 64.6 | 71.3 |
| 55 | 65.3 | 72.2 |
| 56 | 73.1 | 77.6 |
| 57 | 70.7 | 76.5 |
| 58 | 66.2 | 73.7 |
| 59 | 65.0 | 72.2 |
| 60 | 67.9 | 74.8 |
| 61 | 70.1 | 77.6 |
| 62 | 67.5 | 74.2 |
| 63 | 66.9 | 74.1 |
| 64 | 65.6 | 73.1 |
| 65 | 63.3 | 70.6 |
| 66 | 64.1 | 71.7 |
| 67 | 60.7 | 68.6 |
| 68 | 71.2 | 76.6 |
| 69 | 72.5 | 78.7 |
| 70 | 65.4 | 72.3 |
| 71 | 68.0 | 77.2 |
| 72 | 48.3 | 54.2 |
| 73 | 67.5 | 74.7 |
| 74 | 66.9 | 74.4 |
| 75 | 64.8 | 72.7 |
| 76 | 67.5 | 74.9 |
| 77 | 64.9 | 72.8 |

### Example 5: Production of Additional Recombinant Polypeptides for a Nutritional Composition

Additional protein variants of SEQ ID NO: 3 and SEQ ID NO: 50 were produced. Variants include N-terminal truncations, with the truncations ranging in length of 15-158 amino acids. These constructs encode polypeptides comprising the amino acid sequences of SEQ ID NOs: 4-53. Expression of these variants is evaluated in A. oryzae and B. licheniformis. The recombinant polypeptide comprising SEQ ID NO: 52 was highly expressed in B. licheniformis, demonstrating the broad versatility of this protein backbone for use in the food industry.

### Example 6: Production of a Recombinant Polypeptide for Specialized Nutritional Composition

It was desirable to produce a polypeptide comprising at least 105 mg leucine per gram of polypeptide and at least 210 mg of branched chain amino acids per gram of polypeptide for use in nutritional compositions for general nutrition and/or specialized nutrition, such as medical nutrition and/or sports nutrition.

Substitutions were introduced into the parent SEQ ID NO: 51, which is an inactive variant, to produce a polypeptide with a leucine content of 115-137 mg/g polypeptide, and a branched chain amino acids content of 221-250 mg/g polypeptide, and an overall amino acid score of >1. These variants are shown in Table 8.

TABLE 8
Polypeptide variants for general nutrition

| SEQ ID | Leucine | Branched amino acids |
|---|---|---|
| NO. | mg/g protein | mg/g protein |
| 78 | 115 | 221 |
| 79 | 117 | 223 |
| 80 | 119 | 225 |
| 81 | 121 | 227 |
| 82 | 123 | 229 |
| 83 | 125 | 231 |
| 84 | 127 | 233 |
| 85 | 129 | 235 |
| 86 | 131 | 237 |
| 87 | 133 | 239 |
| 88 | 135 | 241 |
| 89 | 137 | 243 |
| 90 | 127 | 237 |
| 91 | 129 | 239 |
| 92 | 131 | 241 |
| 93 | 133 | 243 |
| 94 | 135 | 245 |
| 95 | 137 | 247 |
| 96 | 127 | 241 |
| 97 | 129 | 243 |
| 98 | 131 | 245 |
| 99 | 133 | 247 |
| 100 | 135 | 249 |
| 101 | 137 | 250 |

1. A nutritional composition suitable as a complete protein source for Phenylketonuria (PKU) patients, wherein the composition comprises a recombinant polypeptide which has a nutritionally complete amino acid profile for PKU patients.

2. The nutritional composition of claim 1, wherein the composition does not comprise added free amino acids.

3. The nutritional composition of claim 1, wherein the recombinant polypeptide comprises no phenylalanine residues.

4. The nutritional composition of claim 1, wherein the Amino Acid Score (AAS) of the polypeptide is equal to or greater than 0.94, aside from phenylalanine.

5. The nutritional composition of claim 1, wherein the recombinant polypeptide comprises:
a) at least 50 mg threonine per gram polypeptide
b) at least 9 mg tryptophan per gram polypeptide
c) at least 55 mg tyrosine per gram polypeptide; and
d) at least 66 mg leucine per gram polypeptide.

6. The nutritional composition of claim 1, wherein the recombinant polypeptide comprises an amino acid sequence which has at least 60%, at least 65%, at least 70%, at least 75%, at least 80%, at least 85%, at least 90%, at least 95%, at least 96%, at least 97%, at least 98%, at least 99% sequence identity, or at least 100% sequence identity, to the amino acid sequence of any one of SEQ ID NOs: 2 to 48.

7. The nutritional composition of claim 1, further comprising vitamins and minerals in amounts sufficient to meet daily requirements.

8. The nutritional composition of claim 1, further comprising a carbohydrate source and/or a fat source.

9. The nutritional composition of claim 1, wherein the composition is a tablet, dispersible powder, granule, capsule, liquid, suspension, or syrup.

10. A nutritional composition suitable as a complete protein source, wherein the composition comprises a recombinant polypeptide which has an AAS greater than or equal to 0.94 and the recombinant polypeptide comprises at least 132 mg leucine per gram polypeptide.

11. The nutritional composition of claim 10, wherein the recombinant polypeptide further comprises at least 230 mg branched chain amino acids per gram polypeptide.

12. The nutritional composition of claim 10, further comprising vitamins and minerals in amounts sufficient to meet daily requirements.

13. The nutritional composition of claim 10, further comprising a carbohydrate source and/or a fat source.

14. The nutritional composition of claim 10, wherein the composition is a tablet, dispersible powder, granule, capsule, liquid, suspension, or syrup.

15. A polypeptide comprising the amino acid sequence of SEQ ID NOs: 2 to 48.

16. An isolated polynucleotide encoding the polypeptide of claim 15.

17. A nucleic acid construct or expression vector comprising the polynucleotide of claim 16.

18. A recombinant host cell transformed with the polynucleotide of claim 16.

19. A method of producing a polypeptide, comprising: cultivating the recombinant host cell of claim 18 under conditions suitable for expression of the polypeptide; and recovering the polypeptide.


SEQUENCE LISTING (101 sequences)

SEQ ID NO: 1 - DNA, 1533 bp, Bacillus subtilis
  source 1..1533: mol_type = other DNA
atgaagcttcttccctccttgattggcctggccagtctggcgtccctcgccgtcgccgccatcggcgtgcctggaggcgtcgtggaggcacacatctcgcaggaccagaaccagcccattatcgcgacgtacgaagatgcatccattgaaggatacggcatcgaaaagcgtgatgaggcaaccgcgaaggccatggatgacctctacgacggaaagggctacatctcctattactatgaagaggatccgaacgcaacagtccctcccggatccgcgactgtccaggtcaaggcaccggagactggtctctacgagctctcgctcggatactatatccctgaaggcaacggcgataagcgaacctccatccaggtgaacggctccggagccggtgagttgaccctctccgcacctgcacctggcacagtgcgtaccgaaaagaagatgaccaaggtgatcctcaactcgggtaacaaccgaatccggatcttgaggggctggggatactatggtatcgaacatatcaagttggagagggtcgatgcgaggattcctccggacaaaacaatgatcgatcccttgaacaacccgaaggcgtccaggcaggcgaaggcattgatgaaatacatgacaaaccagtacggagataagatcatctccggacagcagaccctggaggatgcagcctggatctaccgagagaccggaaaatggcctgccttggtctcgtcggacttgatggactactcgccgtcgcgagtcgaaaacggagccacgtcgaacgaggtcgagaagatgattgagtggtacaaggcaggcggaatcgtcggcctctcgtggcactggaacgcacccaagggtattggaggcaacgaaccgggttgtgagtggtggaaaggctactacacagagtgtactacttacgacgtggagtacgcactcaaccatccctggtcggaagactacaaattgttgatccgggatattgacgccatcgcgacgcagctcaagcgtctccaggagcaccacatccccgtcttgtggaggcctctccatcaggcagaaggcggttggtactggtggggagcgaaaggacccgaacctgcgaagaagttgtaccgtctcatgtacgaacggttgacagaaaagcacaacttgaacaacctcatttgggtctggaactccgtccaggaagagtggtatcctggccgtgatgtcgtggacatggtgtcggtcgatatctacaaccctcctggcgactattcccccaacatcgccatgtatatggagctcacctccctctccaagcacaaaaagctcgtcgccttggcggaaaacggacccattcccgaccccgatctcctccagacatatggagcccactggtcgtactacaacacatggacaggcgatgtcttgcgagatggcaagaccaacacaaaggagcatctcaagaaagtgtacaaccacgactatgtcatcactctcgatgaattgcctaagggattgtatgaatcccctcgatggcggtag

SEQ ID NO: 2 - protein, 510 aa, Bacillus subtilis
MKLLPSLIGLASLASLAVAAIGVPGGVVEAHISQDQNQPIIATYEDASIEGYGIEKRDEATAKAMDDLYDGKGYISYYYEEDPNATVPPGSATVQVKAPETGLYELSLGYYIPEGNGDKRTSIQVNGSGAGELTLSAPAPGTVRTEKKMTKVILNSGNNRIRILRGWGYYGIEHIKLERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTYDVEYALNHPWSEDYKLLIRDIDAIATQLKRLQEHHIPVLWRPLHQAEGGWYWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 3 - protein, 491 aa, Synthetic construct
AIGVPGGVVEAHISQDQNQPIIATYEDASIEGYGIEKRDEATAKAMDDLYDGKGYISYYYEEDPNATVPPGSATVQVKAPETGLYELSLGYYIPEGNGDKRTSIQVNGSGAGELTLSAPAPGTVRTEKKMTKVILNSGNNRIRILRGWGYYGIEHIKLERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTYDVEYALNHPWSEDYKLLIRDIDAIATQLKRLQEHHIPVLWRPLHQAEGGWYWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 4 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHQAEGGWYWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 5 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHQAEGGWYWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 6 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHQAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 7 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHQAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 8 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHQAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 9 - protein, 334 aa, Synthetic construct
AERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHQAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 10 - protein, 226 aa, Synthetic construct
PKGIGGNEPGCEWWKGTYTECTTTDVEYALNHPWSEDYKLLIRDIDAIATQLKRLQEHHIPVLWRPLHQAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 11 - protein, 333 aa, Synthetic construct; identical to SEQ ID NO: 7

SEQ ID NO: 12 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 13 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 14 - protein, 333 aa, Synthetic construct; identical to SEQ ID NO: 13

SEQ ID NO: 15 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 16 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYTNTWTGDVLRDGKTNTKEHLKKVYNHDYVITLDELPKGLYESPRWR

SEQ ID NO: 17 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 18 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 19 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYTNTWTGDVLRDGKTNTKEHLKKVYNHDYVITLDELPKGLYESPRWR

SEQ ID NO: 20 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 21 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTYDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 22 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 23 - protein, 333 aa, Synthetic construct; identical to SEQ ID NO: 22

SEQ ID NO: 24 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 25 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYTNTWTGDVLRDGKTNTKEHLKKVYNHDYVITLDELPKGLYESPRWR

SEQ ID NO: 26 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYTNTWTGDVLRDGKTNTKEHLKKVYNHDYVITLDELPKGLYESPRWR

SEQ ID NO: 27 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 28 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 29 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYTNTWTGDVLRDGKTNTKEHLKKVYNHDYVITLDELPKGLYESPRWR

SEQ ID NO: 30 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 31 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTYDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 32 - protein, 333 aa, Synthetic construct; identical to SEQ ID NO: 31

SEQ ID NO: 33 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYTNTWTGDVLRDGKTNTKEHLKKVYNHDYVITLDELPKGLYESPRWR

SEQ ID NO: 34 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 35 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 36 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 37 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 38 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYYNTWTGDVLRDGKTNTKEHLKKVYNHDYVITLDELPKGLYESPRWR

SEQ ID NO: 39 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYTNTWTGDVLRDGKTNTKEHLKKVYNHDYVITLDELPKGLYESPRWR

SEQ ID NO: 40 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 41 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 42 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 43 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTTDVEYALNHPWSEDYKLLIRDIDAIATQLKRLQEHHIPVLWRPLHEAEGGWTWWGAKGPEPAKKLYRLMYERLTEKHNLNNLIWVWNSVQEEWYPGRDVVDMVSVDIYNPPGDYSPNIAMYMELTTLSKHKKLVALAENGPIPDPDLLQTYGAHWSYTNTWTGDVLRDGKTNTKEHLKKVYNHDYVITLDELPKGLYESPRWR

SEQ ID NO: 44 - protein, 491 aa, Synthetic construct
AIGVPGGVVEAHISQDQNQPIIATYEDASIEGYGIEKRDEATAKAMDDLYDGKGYISYYYEEDPNATVPPGSATVQVKAPETGLYELSLGYYIPEGNGDKRTSIQVNGSGAGELTLSAPAPGTVRTEKKMTKVILNSGNNRIRILRGWGYYGIEHIKLELVDARIPPDKTIIDPLSNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTYDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTSLSKHKKLVALAENGPIPDPDLLQTYGAHWSYYNTWTGDVLRDGKTNTKEHLKKVYNHDYVITLDELPKGLYESPRWR

SEQ ID NO: 45 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAQNGPIPDPDLLQTYGAHWSYTNTWTGDVLRDGKTNTKEHLKKVYNHDYVITLDELPKGLYESPRWR

SEQ ID NO: 46 - protein, 333 aa, Synthetic construct
ERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAQNGPIPDPDLLQTYGAHWSYTNTWTGDVLRDGKTNTKEHLKKVYNHDYVITLDELPKGLYESPRWR

SEQ ID NO: 47 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGTYTECTTYDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAQNGPIPDPDLLQTYGAHWSYTNTWTGDVLRDGKTNTKEHLKKVYNHDYVITLDELPKGLYESPRWR

SEQ ID NO: 48 - protein, 333 aa, Synthetic construct
ARVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGYYTECTTTDVEYALNHPWSEDYKLLIRDIDAIATQLKRLQEHHIPVLWRPLHEAEGGWYWWGAKGPEPAKKLYRLMYERLTEKHNLNNLIWVWNSVQEEWYPGRDVVDMVSVDIYNPPGDYSPNIAMYMELTTLSKHKKLVALAQNGPIPDPDLLQTYGAHWSYTNTWTGDVLRDGKTNTKEHLKKVYNHDYVITLDELPKGLYESPRWR

SEQ ID NO: 49 - protein, 491 aa, Synthetic construct
AIGVPGGVVEAHISQDQNQPIIATFEDASIEGYGIEKRDEATAKAMDDLYDGKGYISYFFEEDPNATVPPGSATFQVKAPETGLYELSLGYYIPEGNGDKRTSIQVNGSGAGELTLSAPAPGTVRTEKKMTKVILNSGNNRIRILRGWGYYGIEHIKLERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPRWR

SEQ ID NO: 50 - protein, 491 aa, Synthetic construct
AIGVPGGVVEAHISQDQNQPIIATFEDASIEGYGIEKRDEATAKAMDDLYDGKGYISYFFEEDPNATVPPGSATFQVKAPETGLYELSLGYYIPEGNGDKRTSIQVNGSGAGELTLSAPAPGTVRTEKKMTKVILNSGNNRIRILRGWGYYGIEHIKLERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALALNGPIPDPDLLQTYGAHWSYFNTWTGDVLRDGKTNTKEHLKKVYNHDYVITLDELPKGLYESPRWR

SEQ ID NO: 51 - protein, 491 aa, Synthetic construct
AIGVPGGVVEAHISQDQNQPIIATFEDASIEGYGIEKRDEATAKAMDDLYDGKGYISYFFEEDPNATVPPGSATFQVKAPETGLYELSLGYYIPEGNGDKRTSIQVNGSGAGELTLSAPAPGTVRTEKKMTKVILNSGNNRIRILRGWGYYGIEHIKLERVDARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 52 - protein, 340 aa, Synthetic construct
AITVPGFVVELVDARIPPDKTIIDPLSNPKASREAKALMKYMTNQYGDKIISGQQTLEDAAWIYRETGKWPALVSSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 53 - protein, 340 aa, Synthetic construct
AIGVPGGVVELVDARIPPDKTIIDPLSNPTASREAKALMKYMTNQYGDKIISGQQTLEDAAWIYRETGKWPALVSSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 54 - protein, 491 aa, Synthetic construct
AIGVPGGVAEPHTSQDQNQGIIATFEDASIEGYGIEKRDEATAKAMDDLYDGTGYISYFFEEDPKATVPPGSATFQVKAPETGLYELSLGYYIPEGNGDKATEIQVNGSGAGELTLSAPAPGTVRAEKKMTKVLLNSGNNRIQILRGWGYYGIEYIKLERVEARIPTDKTMIDPLNNPKASPQAKALMKYMTNQYGDKIISGQQTLEDAAWIYRQTGKWPALVSSDLMDYSPSRVENGATSNEVEKMIEWYKAGGIVGLSWHWNAPKGIGGNEPGHEWWRGFYTEFTTFDVEYALNHPWSEDYKLLIRDIDAIATQLKRLQEHHIPVLWRPLHEAEGGWFWWGAKGPEPAKKLYRLMYERLTEKHGLNNLIWVWNSVQEEWYPGDDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYDSPRWR

SEQ ID NO: 55 - protein, 491 aa, Synthetic construct
AIGVPGGAVEPHISQDQNQPIIATFEDASLEGYGIEKRDEATAKAMDDLYDGTGYISYFFEEDPNATVPPGSATFQVKAPEAGLYELSLGYYIPEGNGDKRTSIQVNGDGPGELTLSAPAPGTVRTEKKMTKVILNSGNNRIQILRGWGYYGIEHIKLERVEARIPTDKPMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPRWR

SEQ ID NO: 56 - protein, 491 aa, Synthetic construct
VITNPGGVEEPHISQDQNQEIIATFEDASIEGYGIEKRDEATAKAYDDLYDGTGYISYFFEEDPKATVPPGSATFQVKAPETGLYELSLGYYIPEGNGDKRTSIQVNGEGAGELVLSAPPPGTVRAEKKMTKVLLNSGNNRIQILRGWGYYGIEHIKLERVEARIPPDKTIIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPRWR

SEQ ID NO: 57 - protein, 491 aa, Synthetic construct
AIGVPGGAVGPHISQDQNQPIIATFEDASIEGYGIEKRDEATAKAMDDLYDGKGYISYFFEEDPKATVPPGSATFQVKAPETGVYELSLGYYIPEGNGDKRTEIQVNGSGAGELTLSAPAPGTVRAEKKMTKVLLNSGNNRIQILRGWGYYGIEHIKLERVEARIPPDKTIIDPLSNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYDSPRWR

SEQ ID NO: 58 - protein, 474 aa, Synthetic construct
AQNQPIIATFEDASLEGYGIEKRDEATAKAMDDLYDGTGYISYFFEEDPKATVPPGSATFEVKAPETGLYELSLGYYIPEGNGDKLTEIQVNGSGPGELTLSAPAPGTVRAEKKMTKVLLNAGNNRIRILRGWGYYGIEHIKLELVEARIPPDKTIIDPLSNPKASREAKALMKYMTNQYGDKIISGQQTLEDAAWIYRETGKWPALVSSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 59 - protein, 491 aa, Synthetic construct
AIGVPGGAVEPHISQDQNQPIIATFEDASLEGYGIEKRDEATAKAMDDLYDGKGYISYFFEEDPNATVPPGSATFQVKAPEAGLYELSLGYYIPEGNGDKRTSIQVNGDGPGELTLSAPAPGTVRTEKKMTKVILNSGNNRIQILRGWGYYGIEHIKLERVEARIPTDKPMIDPLNNPKASRQAKALMKYMTNQYGDKIISGQQTLEDAAWIYRETGKWPALVSSDLMDYSPSRVENGATSNEVEKMIEWYKVGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPRWR

SEQ ID NO: 60 - protein, 490 aa, Synthetic construct
AIGVPGGVVEAHISQDQNQPIIATFEDASLEGYGIEKRDEATAKAMDDLYDGKGYISYFFEEDPNATVPPGSATFQVKAPETGLYELSLGYYIPEGNGDKRTSIQVNGEGPGELTLSAPAPGTVRTEKKMTKVILNSGNNRIRILRGWGYYGIEHIKLERVERIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLPESPRWR

SEQ ID NO: 61 - protein, 474 aa, Synthetic construct
AQNQPIIQTFEDASLEGYGIEKRDEATAKAMDDLYDGPGYISYFFEEDPKATVPPGSATFQVKAPEAGLYDLSLGYYIPEGNGDKRTEIQVNGDGAGELVLSAPAPGTVRAEKKMTKVLLNSGNNRIQILRGWGYYGIEHIKLERVEARIPPDKTIIDPLSNPKASREAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPS

SEQ ID NO: 62 - protein, 474 aa, Synthetic construct
AQNQPIIATFEDASLEGYGIEKRDEATAKAMDDLYDGTGYISYFFEEDPKATVPPGSATFEVKAPETGLYELSLGYYIPEGNGDKLTEIQVNGSGPGELTLSEPAPGTVRAEKKMTKVLLNAGNNRIRILRGWGYYGIEHIKLELVDARIPPDKTIIDPLSNPKASREAKALMKYMTNQYGDKIISGQQTLEDAAWIYRETGKWPALVSSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 63 - protein, 474 aa, Synthetic construct
AQNQPIIATFEDASLEGYGIEKRDEATAKAMDDLYDGTGYISYFFEEDPKATVPPGSATFEVKAPETGLYELSLGYYIPEGNGDKLTEIQVNGSGIGELTLSAPPPGTVRAEKKMTKVLLNAGNNRIRILRGWGYYGIEHIKLELVEARIPPDKTIIDPLSNPKASREAKALMKYMTNQYGDKIISGQQTLEDAAWIYRETGKWPALVSSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 64 - protein, 489 aa, Synthetic construct
AIGVPGGAVEPHISQDQNQTIIATFEDASLEGYGIEKRDEATAKAMDPLYDGPGYISYFFDEDPNATVPPGSAWFTVSAPENGLYELSLGYYIPEGNGDKATIIQVNGEGAEELTLSAPAPGTVRTEKKMTKVLLNSGRNRIQILRGWGYYGIEHIKLEPVEARIPHDKTMIDPLNNPKATPQAKALMKFMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIAVQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 65 - protein, 489 aa, Synthetic construct
AIGVPGGAVEPEISQDQNQTIIATFEDASLEGYGIEKRDEATAKAMDPLYDGPGYISYFFEEDPNATVPPGSATFTVSAPENGLYELSLGYYIPEGNGDKATIIQVNGEGAVELTLSAPAPGTVRTEKKMTKVLLNSGRNRIQILRGWGYYGIEHIKLEPVEARIPHDKTMIDPLNNPKATPQAKALMKFMTNQYGDKIISGQQTLEDAAWIYRETGKWPALVSSDLMDYSPSRVENGATSNEVEKMIEWYKAGGIVGLSWHWNAPKGIGGNEPGCEWWRGFYTECTTFDVEYALNHPWSEDYKLLIRDIDAIAVQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 66 - protein, 474 aa, Synthetic construct
AQNQPIIATFEDASIEGYGIEKRDEATAKAMDALYDGKGYISYFFEEDPKATVPPGSATFEVKAPETGLYELSLGYYIPEGNGDKATEIQVNGDGAGELYLSAPPPGTVRTEKKMTKVLLNAGNNRIQILRGWGYYGIEHIKLERSDARIPPDKTIIDPLSNPKASREAKALMKYMTNQYGDKIISGQQTLEDAAWIYIETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGSIPDPDLLQTYGAHWSYFNTWTGDVLRDGKTNTKEHLKKVYNHDYVITLDELPKGLYESPR

SEQ ID NO: 67 - protein, 474 aa, Synthetic construct
AQNQTIIATFEDASLEGYGIEKRDEATAKAMDDLYDGPGYISYFFEEDPNATVPPGSATFTVSAPENGLYELSLGYYIPEGNGDKATEIQVNGEGPGELTLSAPAPGTVRTEKKMTKVLLNSGNNRIQILRGWGYYGIEHIKLEPVEARIPNDKTMIDPLNNPKATPQAKALMKFMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIAVQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 68 - protein, 489 aa, Synthetic construct
AIGVPGGAVEPHISQDQNQPIIATFEDASIEGYGIEKRDEATAKMYDDLYDGPGYISYFFEEDPNATVPPGSATFQVKAPEAGLYELSLGYYIPEGNGDKRTSIQVNGEGAGELTLSAPAPGTVRTEKKMTKVILNSGNNRIQILRGWGYYGIEHIKLERVEARIPPDKTIIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 69 - protein, 491 aa, Synthetic construct
AIGVPGGAVEPHISQDQNQPIIATFEDASIEGYGIEKRDEATAKMYDDLYDGPGYISYFFEEDPNATVPPGSATFQVKAPEAGLYELSLGYYIPEGNGDKRTSIQVNGEGAGELTLSAPAPGTVRTEKKMTKVILNSGNNRIQILRGWGYYGIEHIKLERVEARIPPDKTMIDPLNNPKASRQAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPRWR

SEQ ID NO: 70 - protein, 474 aa, Synthetic construct
AQNQPIIATFEDASIEGYGIEKRDEATAKMMDDLYDGTGYISYFFEEDPNATVPPGSATFEVKAPETGLYELSLGYYIPEGNGDKLTEIQVNGLGPGELTLSAPAPGTVRAEKKMTKVLLNKGNNRIRILRGWGYYGIEHIKLELVEARIPPDKTIIDPLSNPKASREAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 71 - protein, 474 aa, Synthetic construct
AQNQPIIATFEDASIEGYGIEKRDEATAKAMDDLYDGKGYISYFFEEDPKATEPPGSATFQVKAPETGVYELSLGYYIPEGNGDKRTEIQVNGSGAGELTLSAPAPGTVRAEKKMTKVLLNSGNNRIQILRGWGYYGIEHIKLERVEARIPPDKTIIDPLSNPKASREAKALMKYMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIATQLKRLQEHHIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDFLRDGKTNTKEHLKKVYNHDYVITLDELPKGLYDSPR

SEQ ID NO: 72 - protein, 474 aa, Synthetic construct
AQNQPIIATFEDASLEGYGIEKRDEATAKAMDDLYDGTGYISYFFEEDPKATVPPGSATFEVKAPETGLYELSLGYYIPEGNGDKLTEIQVNGSGPGELTLSAPAPGTVRAEKKMTKVLLNAGNNRIRILRGWGYYGIEHIKLELVEARIPPDKTIIDPLSNPKASREAKALMKYMTNQYGDKIISGQQTLEDAAWIYRETGKWPALVRSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 73 - protein, 474 aa, Synthetic construct
AQNQPIIATFEDASLEGYGIEKRDEATAKAMDPLYDGTGYISYFFEEDPKATVPPGSATFTVKAPENGLYELSLGYYIPEGNGDKLTEIQVNGSGPGELTLSEPAPGTVRAEKKMTKVLLNAGNNRIRILRGWGYYGIEHIKLELVDARIPPDKTIIDPLSNPKASREAKALMKYMTNQYGDKIISGQQTLEDAAWIYRETGKWPALVSSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 74 - protein, 474 aa, Synthetic construct
AQNQPIIATFEDASLEGYGIEKRDEATAKAMDPLYDGTGYISYFFEEDPKATVPPGSATFEVKAPETGLYELSLGYYIPEGNGDKLTEIQVNGSGPGELTLSEPAPGTVRAEKKMTKVLLNAGNNRIRILRGWGYYGIEHIKLELVDARIPPDKTIIDPLSNPKATREAKALMKYMTNQYGDKIISGQQTLEDAAWIYRETGKWPALVSSDLMDYSPSRVENGATSNEAEKMIEWYKAGGIVGLSWHWNAPKGIGGNEPGCEWWKGFYTECTTFDVEYALNHPWSEDYKLLIRDIDAIATQLKRLQEHKIPVLWRPLHEAEGGWFWWGAKGPEPAKKLYRLMYERLTEKHNLNNLIWVWNSVQEEWYPGHDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 75 - protein, 489 aa, Synthetic construct
AIGVPGGAVEPHISQDQNQTIIATFEDASLEGYGIEKRDEATAKMMDPLYDGPGYISYFFDEDPNATVPPGSAWFTVSAPENGLYELSLGYYIPEGNGDKATIIQVNGEGAEELVLSAPAPGTVRTEKKMTKVLLNKGRNRIQILRGWGYYGIEHIKLEPVEARIPHDKTMIDPLNNPKATPQAKALMKFMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIAVQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHRKLVALAENGPIPDPDLLQTYGAHWSYFNTWTGDVLRDGKTNTKEHLKKVYNHDYVITLDELPKGLYESPR

SEQ ID NO: 76 - protein, 489 aa, Synthetic construct
AIGVPGGAVEPHISQDQNQTIIATFEDASLEGYGIEKRDEATAKMMDPLYDGPGYISYFFDEDPNATVPPGSAWFTVSAPENGLYELSLGYYIPEGNGDKATIIQVNGEGAEELVLSAPAPGTVRTEKKMTKVLLNSGRNRIQILRGWGYYGIEHIKLEPVEARIPPDKTMIDPLNNPKATPQAKALMKFMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIAVQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 77 - protein, 489 aa, Synthetic construct
AIGVPGGAVEPHISQDQNQTIIATFEDASLEGYGIEKRDEATAKKMDPLYDGPGYISYFFDEDPNATVPPGSAWFTVSAPENGLYELSLGYYIPEGSGDKLTIIQVNGEGAEELTLSAPAPGTVRAEKKMTKVLLNSGRNRIQILRGWGYYGIEHIKLEPVEARIPHDKTMIDPLNNPKATPQAKALMKFMTNQYGDKIISGQQTLEDAAWIYRETGKWPALVSSDLMDYSPSRVENGATSNEVEKMIEWYKAGGIVGLSWHWNAPKGIGGNEPGCEWWKGFYTECTTFDVEYALNHPWSEDYKLLIRDIDAIAVQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAENGPIPDPDLLQTYGAHWSYFNTWTGDVLRDGKTNTKEHLKKVYNHDYVITLDELPKGLYESPR

SEQ ID NO: 78 - protein, 491 aa, Synthetic construct
AILVPGGVVEAHISQDQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNPKASRQAKALMKYMTNQYGDKIISGQQTLEDAAWIYRETGKWPALVSSDLMDYSPSRVENGATSNEVEKMIEWYKALGIVGLSWHWNAPKLIGGNEPGCEWWKGFYTECTTFDVEYALNHPWSELYKLLIRDIDAILTQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 79 - protein, 491 aa, Synthetic construct
AILVPGGVVEAHISQDQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNPKASRQAKALMKYMTNQYGDKIISGQQTLEDAAWIYRETGKWPALVSSDLMDYSPSRVENGATSNEVEKMIEWYKALGIVGLSWHWNAPKLIGGNEPGCEWWKGFYTECTTFDVEYALNHPWSELYKLLIRDILAILTQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 80 - protein, 491 aa, Synthetic construct
AILVPGGVVEAHISQDQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNPKASRQAKALMKYMTNQYGDKIISGQQTLEDAAWIYRETGKWPALVSSDLMDYSPSRVENGATSNEVEKMIEWYKALGIVGLSWHWNAPKLIGGLEPGCEWWKGFYTECTTFDVEYALNHPWSELYKLLIRDILAILTQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 81 - protein, 491 aa, Synthetic construct
AILVPGGVVEAHISQDQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTNQYGDKIISGQQTLEDAAWIYRETGKWPALVSSDLMDYSPSRVENGATSNEVEKMIEWYKALGIVGLSWHWNAPKLIGGLEPGCEWWKGFYTECTTFDVEYALNHPWSELYKLLIRDILAILTQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 82 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTNQYGDKIISGQQTLEDAAWIYRETGKWPALVSSDLMDYSPSRVENGATSNEVEKMIEWYKALGIVGLSWHWNAPKLIGGLEPGCEWWKGFYTECTTFDVEYALNHPWSELYKLLIRDILAILTQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 83 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLQYGDKIISGQQTLEDAAWIYRETGKWPALVSSDLMDYSPSRVENGATSNEVEKMIEWYKALGIVGLSWHWNAPKLIGGLEPGCEWWKGFYTECTTFDVEYALNHPWSELYKLLIRDILAILTQLKRLQEHHIPVLWRPLHEAEGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 84 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLQYGDKIISGQQTLEDAAWIYRETGKWPALVSSDLMDYSPSRVENGATSNEVEKMIEWYKALGIVGLSWHWNAPKLIGGLEPGCEWWKGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 85 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLQYGDKIISGQQTLEDAAWIYRETGKWPALVSSDLMDYSPSRVENGATSNEVEKMIEWYKALGIVGLSWHWLAPKLIGGLEPGCEWWKGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 86 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLQYGDKIISGQQTLEDAAWIYRETGKWPALVSSDLMDYSPSRVENGATSNEVEKMIEWYKALGIVGLSWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 87 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLQYGDKIISGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 88 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLQYGDKIISGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWLYFNTWTGDVLRDGKTNTKEHLKKVYNHDYVITLDELPKGLYESPRWR

SEQ ID NO: 89 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKLLMKYMTLQYGDKIISGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYPGRDVVDMVSVDIYNPPGDYSPNIAMYMELTFLSKHKKLVALAQNGPIPDPDLLQTYGAHWLYFNTWTGDVLRDGKTNTKEHLKKVYNHDYVITLDELPKGLYESPRWR

SEQ ID NO: 90 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIISGQQTLEDAAWIYRETGKWPALVSSDLMDYSPSRVENGATSNEVEKMIEWYKALGIVGLSWHWNAPKLIGGLEPGCEWWKGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 91 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIISGQQTLEDAAWIYRETGKWPALVSSDLMDYSPSRVENGATSNEVEKMIEWYKALGIVGLSWHWLAPKLIGGLEPGCEWWKGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 92 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIISGQQTLEDAAWIYRETGKWPALVSSDLMDYSPSRVENGATSNEVEKMIEWYKALGIVGLSWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 93 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIISGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 94 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIISGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWLYFNTWTGDVLRDGKTNTKEHLKKVYNHDYVITLDELPKGLYESPRWR

SEQ ID NO: 95 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKLLMKYMTLVYGDKIISGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHEALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWLYFNTWTGDVLRDGKTNTKEHLKKVYNHDYVITLDELPKGLYESPRWR

SEQ ID NO: 96 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIIVGQQTLEDAAWIYRETGKWPALVSSDLMDYSPSRVENGATSNEVEKMIEWYKALGIVGLSWHWNAPKLIGGLEPGCEWWKGFYTECTTFDVEYALNHPWSELYKLLIRDILAILTQLKRLQEHHIPVLWRPLHVALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 97 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIIVGQQTLEDAAWIYRETGKWPALVSSDLMDYSPSRVENGATSNEVEKMIEWYKALGIVGLSWHWLAPKLIGGLEPGCEWWKGFYTECTTFDVEYALNHPWSELYKLLIRDILAILTQLKRLQEHHIPVLWRPLHVALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 98 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIIVGQQTLEDAAWIYRETGKWPALVSSDLMDYSPSRVENGATSNEVEKMIEWYKALGIVGLSWHWLAPKLIGGLEPGCEWWLGFYTECTTFDVEYALNHPWSELYKLLIRDILAILTQLKRLQEHHIPVLWRPLHVALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 99 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIIVGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHVALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWSYFNTWTGDVLRDGKTNTKEHLKKVYNHDYVITLDELPKGLYESPRWR

SEQ ID NO: 100 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKALMKYMTLVYGDKIIVGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHVALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWLYFNTWTGDVLRDGKTNTKEHLKKVYNHDYVITLDELPKGLYESPRWR

SEQ ID NO: 101 - protein, 491 aa, Synthetic construct
AILVPGGVVEAHISQLQNQLIIATFEDASIEGYGIEKRDEATAKAMDDLYDGKGYISYFFEEDPNATVPPLLATFQVKLPETGLYLLSLGYYIPLGNGDKRTSIQVNGLGLLLLTLSAPALGTVRTLKKMTKVILNSGNNRIRILRGWGYYGIEHIKLELVDLRIPPDKTMIDPLNNLKASRQAKLLMKYMTLVYGDKIIVGQQTLEDAAWIYRETGKWPALVSSDLMDYSPSRVENGATSNEVEKMIEWYKALGIVGLLWHWLAPKLIGGLEPGCEWWLGFYTECTTFDVEYALNHPWSELYKLLIRDILAILTQLKRLQEHHIPVLWRPLHVALGGWFWWGAKGPEPAKKLYRLMYERLTEKHNLNNLIWVWNSVQEEWYVGRDVVDMVSVDIYNPPGDYSPNIAMYMELTFLSKHKKLVALAQNGPIPDPDLLQTYGAHWLYFNTWTGDVLRDGKTNTKEHLKKVYNHDYVITLDELPKGLYESPRWR